SEQUENCE LISTING (20 sequences)

SEQ ID NO: 1 - DNA, 2803 bp, Artificial Sequence
  Description of Artificial Sequencedouble stranded circular Plasmid A
tcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggaacgtagcgtaccggtgcaaaaagcaaaaaggctcgacgccgcaaaaagacaaaaaggctcgacgccgcaaaaagtacaaaaaggctcgacgccgcagctcgtcctagctagcgtcgtagcgtcttaag

SEQ ID NO: 2 - DNA, 2762 bp, Artificial Sequence
  Description of Artificial Sequencedouble stranded circular Plasmid B
ggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagatgccgctaagtactatgccgctaactactatgccgctaattactatgccgctaaatactatgccgctaactagtatgccgctatgca

SEQ ID NO: 3 - DNA, 4176 bp, Artificial Sequence
  Description of Artificial Sequencedouble stranded circular Plasmid C, plasmid pTrc99a
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattcgagctcggtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 4 - DNA, 351 bp, Artificial Sequence
  Description of Artificial SequenceEcoRI/PvuII restriction fragment from Plasmid A
aattcgagctcggtacccgggaacgtagcgtaccggtgcaaaaagcaaaaaggctcgacgccgcaaaaagacaaaaaggctcgacgccgcaaaaagtacaaaaaggctcgacgccgcagctcgtcctagctagcgtcgtagcgtcttaagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccag

SEQ ID NO: 5 - DNA, 310 bp, Artificial Sequence
  Description of Artificial SequenceEcoRI/PvuII restriction fragment from Plasmid B
aattcgagctcggtacccggggatcctctagatgccgctaagtactatgccgctaactactatgccgctaattactatgccgctaaatactatgccgctaactagtatgccgctatgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccag

SEQ ID NO: 6 - DNA, 352 bp, Artificial Sequence
  Description of Artificial SequenceEcoRI/PvuII restriction fragment from Plasmid C
ctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaatt

SEQ ID NO: 7 - DNA, 12 bp, Artificial Sequence
  Description of Artificial Sequencetarget sequence in EcoRI/PvuII restriction fragment of Plasmid A
aaaaagcaaaaa

SEQ ID NO: 8 - DNA, 13 bp, Artificial Sequence
  Description of Artificial Sequencetarget sequence in EcoRI/PvuII restriction fragment of Plasmid A
aaaaagacaaaaa

SEQ ID NO: 9 - DNA, 14 bp, Artificial Sequence
  Description of Artificial Sequencetarget sequence in EcoRI/PvuII restriction fragment of Plasmid A
aaaaagtacaaaaa

SEQ ID NO: 10 - DNA, 11 bp, Artificial Sequence
  Description of Artificial Sequencetarget sequence in EcoRI/PvuII restriction fragment of Plasmid C
tgacaattaat

SEQ ID NO: 11 - DNA, 12 bp, Artificial Sequence
  Description of Artificial Sequencetarget sequence in EcoRI/PvuII restriction fragment of Plasmid C
gacaattaatca

SEQ ID NO: 12 - DNA, 10 bp, Artificial Sequence
  Description of Artificial Sequencetarget sequence in EcoRI/PvuII restriction fragment of Plasmid C
aattaatcat

SEQ ID NO: 13 - DNA, 68 bp, Artificial Sequence
  Description of Artificial Sequencefirst set 5′-phosphorylated complementary oligonucleotide for Plasmid A
ccgggaacgtagcgtaccggtgcaaaaagcaaaaaggctcgacgccgcaaaaagacaaaaaggctcga

SEQ ID NO: 14 - DNA, 68 bp, Artificial Sequence
  Description of Artificial Sequencefirst set 5′-phosphorylated complementary oligonucleotide for Plasmid A
ggcgtcgagcctttttgtctttttgcggcgtcgagcctttttgctttttgcaccggtacgctacgttc

SEQ ID NO: 15 - DNA, 65 bp, Artificial Sequence
  Description of Artificial Sequencesecond set 5′-phosphorylated complementary oligonucleotide for Plasmid A
gccgcaaaaagtacaaaaaggctcgacgccgcagctcgtcctagctagcgtcgtagcgtcttaag

SEQ ID NO: 16 - DNA, 65 bp, Artificial Sequence
  Description of Artificial Sequencesecond set 5′-phosphorylated complementary oligonucleotide for Plasmid A
cgacttaagacgctacgacgctagctaggacgagctgcggcgtcgagcctttttgtactttttgc

SEQ ID NO: 17 - DNA, 56 bp, Artificial Sequence
  Description of Artificial Sequencefirst set 5′-phosphorylated complementary oligonucleotide for Plasmid B
ctagatgccgctaagtactatgccgctaactactatgccgctaattactatgccgc

SEQ ID NO: 18 - DNA, 48 bp, Artificial Sequence
  Description of Artificial Sequencefirst set 5′-phosphorylated complementary oligonucleotide for Plasmid B
catagtaattagcggcatagtagttagcggcatagtacttagcggcat

SEQ ID NO: 19 - DNA, 36 bp, Artificial Sequence
  Description of Artificial Sequencesecond set 5′-phosphorylated complementary oligonucleotide for Plasmid B
taaatactatgccgctaactagtatgccgctatgca

SEQ ID NO: 20 - DNA, 36 bp, Artificial Sequence
  Description of Artificial Sequencesecond set 5′-phosphorylated complementary oligonucleotide for Plasmid B
tagcggcatactagttagcggcatagtatttagcgg

(19) United States
(12) Patent Application Publication
(10) Pub. No.: US 2003/0199516 A1
(43) Pub. Date: Oct. 23, 2003

(54) Methods of treating infection by drug resistant bacteria

(75) Inventors: Heinz E. Moser, San Mateo, CA (US); Eldon E. Baird, Half Moon Bay, CA (US); Roland W. Burli, San Francisco, CA (US); Yigong Ge, So. San Francisco, CA (US); Sarah White, Half Moon Bay, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Genesoft, Inc., San Francisco, CA

(21) Appl. No.: 10/244,142

(22) Filed: Sep. 12, 2002

Related U.S. Application Data
(60) Provisional application No. 60/322,704, filed on Sep. 13, 2001.

(51) Int. Cl. A61K 31/501; A61K 31/497; A61K 31/444; A61K 31/4709; A61K 31/427; A61K 31/422; A61K 31/4178; A61K 31/381; A61K 31/4196; C07D 409/02; C 07D 4 3/14; C07D 417/14; C07D 413/14

(52) U.S. Cl. 514/252.02; 514/355; 514/341; 514/365; 514/372; 514/397; 514/406; 514/422; 514/444; 544/238; 546/255; 546/256; 546/148; 548/202; 548/215; 548/312.7; 548/365.1; 544/405; 548/527; 548/518; 549/49; 549/59; 514/255.05

(57) ABSTRACT

Methods are provided for treating an infection by Gram-positive bacteria in a mammal, by administering to the mammal an effective amount of a compound that binds noncovalently in the minor groove of duplex DNA, the compound being identified by a number of DNA binding parameters and, in many instances, being a polyaromatic compound.

### CROSS-REFERENCES TO RELATED APPLICATIONS

This application claims the benefit of U.S. Ser. No. 60/322,704 filed Sep. 13, 2001; the disclosures of which is incorporated herein by reference

### STATEMENT AS TO RIGHTS TO INVENTIONS MADE UNDER FEDERALLY SPONSORED RESEARCH AND DEVELOPMENT

The United States government may have certain rights to this invention under DARPA Grant No. N65236-99-1-5427.

### REFERENCE TO A “SEQUENCE LISTING,” A TABLE, OR A COMPUTER PROGRAM LISTING APPENDIX SUBMITTED ON A COMPACT DISK.

### BACKGROUND OF THE INVENTION

Many compounds, either naturally occurring or synthetic, have been found to bind to double stranded nucleic acid, especially double stranded deoxyribonucleic acid (“dsDNA”). Depending on their structure, the compounds bind to different parts of the nucleic acid. Some bind to the major groove while others associate with the minor groove. Still others intercalate between adjacent base pairs. Combination binding modes are also known, in which a compound has binding interactions with more than one site in the nucleic acid.

Certain dsDNA binding compounds can be used to regulate the expression of genes for medical purposes. If a disease is characterized by the overexpression or the undesired expression of a gene (e.g., an oncogene), the disease may be treated by suppressing in toto or in part the expression of the gene by the binding of such compounds to the gene or a promoter site thereof. Infections by pathogens such fungi, bacteria, and viruses may be combated with compounds that affect the expression of genes essential for the proliferation or existence/survival of the pathogen.

Whatever the application, the compound must strongly bind to dsDNA, generally meaning that it binds with an association constant of at least 10⁶ M⁻¹, preferably at least about 10⁹ M⁻¹. However, binding strength alone is not determinative of efficaciousness, whether in a therapeutic, anti-infective, or other applications. Many other factors come into play, including, for instance, cellular uptake, stability, toxicity, binding specificity, and the like. A compound that is acceptable or superior in one characteristic may be fatally deficient in another characteristic. Thus, there is a continuing need to develop new classes of nucleic acid binding compounds for use in such applications.

### BRIEF SUMMARY OF THE INVENTION

In one aspect, the present invention provides a method for treating an infection by gram-positive bacteria in a mammal, by administering to the mammal an effective amount of a compound that binds noncovalently in the minor groove of duplex DNA, which compound:

i) binds with a dissociation constant of equal to or less than 100 nM to at least one of:

(a) a target sequence AAAAAGCAAAAA in the 351 base pair EcoRI/PvuII restriction fragment of a polynucleotide of SEQ ID NO. I;

(b) a target sequence AAAAAGACAAAAA in the 351 base pair EcoRI/PvuII restriction fragment of a polynucleotide of SEQ ID NO. I;

(c) a target sequence AAAAAGTACAAAAA in the 351 base pair EcoRI/PvuII restriction fragment of a polynucleotide of SEQ ID NO. I;

(d) a target sequence AGTACT in the 310 base pair EcoRI/PvuII restriction fragment of a polynucleotide of SEQ ID NO. II;

(e) a target sequence AATACT in the 310 base pair EcoRUPvuII restriction fragment of a polynucleotide of SEQ ID NO. II;

(f) a target sequence ATTACT in the 310 base pair EcoRI/PvuII restriction fragment of a polynucleotide of SEQ ID NO. II;

(g) a target sequence TGACAATTAAT in the 352 base pair EcoRI/PvuII restriction fragment of a polynucleotide of SEQ ID NO. III;

(h) a target sequence GACAATTAATCA in the 352 base pair EcoRI/PvuII restriction fragment of a polynucleotide of SEQ ID NO. III;

(i) a target sequence AATTAATCAT in the 352 base pair EcoRI/PvuII restriction fragment of a polynucleotide of SEQ ID NO. III;

a target sequence ACAATTA in the 352 base pair EcoRI/PvuII restriction fragment of a polynucleotide of SEQ ID NO. III; and

(k) a target sequence ACAATTAAT in the 352 base pair EcoRI/PvuII restriction fragment of a polynucleotide of SEQ ID NO. III;

ii) exhibits a MIC of less than or equal to 2 μg/mL against at least one of Enterococcus faecium ATCC 51559, Staphylococcus aureus ATCC 27660, Staphylococcus aureus ATCC 33591, Staphylococcus aureus ATCC 43300, and Streptococcus pneumoniae ATCC 51422;

iii) exhibits a MIC of greater than or equal to 32 μg/mL against Candida albicans ATCC 38247; and

iv) has a molecular weight of from 100 to about 1100.

In a related aspect, the present invention provides methods as above, wherein the compound has activity ratio X/Y equal to or greater than 16, wherein X is the MIC of the compound against Candida albicans ATCC 38247 and Y is the lowest MIC of the compound from among the MIC's for Enterococcus faecium ATCC 51559, Staphylococcus aureus ATCC 27660, Staphylococcus aureus 33591, Staphylococcus aureus ATCC 43300, and Streptococcus pneumoniae ATCC 51422.

In yet another aspect, the present invention provides a compound useful for the treatment of a bacterial infection, the compound having the formula:

A-((L¹)ₚ—Ar¹)ₙ-L^{x}-B (I)

wherein A is a substituted or unsubstituted aryl or heteroaryl group, a substituted or unsubstituted heterocyclic group, an unsubstituted amino group or a mono- or di-alkyl amino group; the subscript n is an integer of from 2 to 7; the subscript p in each instance is an integer of from 0 to 1, indicating the presence or absence of each linking group (L¹); each L¹ is a linking group in which the superscript i is an integer of from 1 to n, and each linking group can be the same or different from the other linking groups and is selected from the group consisting of —NH—, —NR—, —CONH—, —SO₂NH—, —CONR—, —SO₂NR—, (C₁-C₆)alkylene, (C₁-C₆)heteroalkylene, and combinations thereof in which each R is independently (C₁-C₆)alkyl; Ar¹ is a substituted or unsubstituted aryl or heteroaryl group, in which the superscript i is an integer of from 1 to n and denotes the position away from A that is occupied by each aryl or heteroaryl group, and each Ar group can be the same or different from any other Ar group; L^{x} is a linking group selected from —NH—, —NR—, —CONH—, —SO₂NH—, —CONR—, —SO₂NR—, (C₁-C₆)alkylene, (C₁-C₆)heteroalkylene, and combinations thereof; and B is a member selected from a substituted or unsubstituted aryl or heteroaryl group, a substituted or unsubstituted heterocyclic group, and an unsubstituted amino group or a mono- or di-alkyl amino group. Additionally, the compounds of the invention:

i) bind with a dissociation constant of equal to or less than 100 nM to at least one of the target sequences noted above in (a) through (k);

ii) exhibit a MIC of less than or equal to 2 μg/mL against at least one of Enterococcus faecium ATCC 51559, Staphylococcus aureus ATCC 27660, Staphylococcus aureus ATCC 33591, Staphylococcus aureus ATCC 43300, and Streptococcus pneumoniae ATCC 51422;

iii) exhibit a MIC of greater than or equal to 32 μg/mL against Candida albicans ATCC 38247; and

iv) have a molecular weight of from 100 to about 1100.

These and other features of the present invention will be apparent to one of skill in the art upon reading the complete disclosure.

### BRIEF DESCRIPTION OF THE DRAWINGS

FIGS. 1-4 illustrate structures of compounds useful in the present invention.

FIGS. 5-7 illustrate maps of plasmids used in DNA binding protocols.

### DETAILED DESCRIPTION OF THE INVENTION

Abbreviations and Definitions

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)metlhyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propyynyl, 3-butynyl, and the higher homologs and isomers.

The term “alkylene” by itself or as part of another substituent means a divalent radical derived from an alkane, as exemplified by —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having six or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH)—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified by —CH₂—CH₂—S—CH₂CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include trifluoromethyl, 2,2,2-trifluoroethyl, 4-chlorobutyl, 3-bromopropyl, and the like.

The term “aryl” means, unless otherwise stated, a polyunsaturated, typically aromatic, hydrocarbon substituent which can be a single ring or multiple rings (up to three rings) which are fused together or linked covalently. The term “heteroaryl” refers to aryl groups (or rings) that contain from zero to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquilnolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NB, —NH—C(NH₂)═NR′, —S(O)R′, —S(O)₂R′, —S(O)₂NR′R″, —CN and —NO₂ in a number ranging from zero to (2 m′+1), where m is the total number of carbon atoms in such radical. R′, R″ and R′″ each independently refer to hydrogen, unsubstituted (C₁-C₈)alkyl and heteroalkyl, unsubstituted aryl, aryl substituted with 1-3 halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like). Preferably, the substituted alkyl and heteroalkyl groups have from 1 to 4 substituents, more preferably 1, 2 or 3 substituents. Exceptions are those perhalo alkyl groups (e.g., pentafluoroethyl and the like) which are also preferred and contemplated by the present invention.

Similarly, substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

As used herein, the term “heteroatom” is meant to include oxygen (O), nitrogen (N), sulfur (S) and silicon (Si).

The term “pharmaceutically acceptable salts” is meant to include salts of the active compounds which are prepared with relatively nontoxic acids or bases, depending oil the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

In the discussions below, reference is made to dsDNA as the nucleic acid, but it is to be understood that the invention is not limited to dsDNA and is applicable to other nucleic acids, i.e., ribonucleic acid.

General

Over the last two decades, there has been a shift in the hospital environment from a predominance of Gram-negative bacteria to Gram-positive bacteria, such that Gram-positive bacteria currently account for about 70% of the bloodstream infections in hospitalized patients. Such a change in predominance has been reported not only in the United States, but also in Europe and is probably a worldwide phenomenon.

Further, there has been an alarming increase in resistance of various bacterial strains to different classes of antibiotics; e.g., in some hospitals, 50% of S. aureus infections are caused by methicillin-resistant strains, most of which are also resistant to macrolides and quilnolones. In these cases, patients must be treated with the glycopeptide vancomycin.

Usage of large quantities of vancomycin has led to the emergence of S. aureus strains with reduced susceptibility to the drug. In addition, Enterococci with high levels of vancomycin resistance now constitute a major challenge. Accordingly, there is an increasing need for new antibiotics with activity against Gram-positive bacteria, particularly for agents with a novel mechanism of action and without cross-resistance to known drugs.

The present invention derives from the surprising discovery that a number of compounds found to interact predominantly in the minor groove of bacterial DNA share certain common structural as well as functional features, in particular bactericidal activity against Gram-positive bacteria. Accordingly, assays have been developed to screen for such compounds and methods are provided for the use of such compounds.

Without being bound by theory, compounds provided herein are believed to bind to a target bacteria's DNA, in particular its minor groove. Double-helical DNA (also referred to as double-stranded DNA, B-DNA, or beta-DNA) has two helical grooves running along its length, with the edges of the bases forming the groove floors and the sugar and phosphate backbone residues forming the groove walls. The asymmetry of the backbone residues leads to the two grooves being of unequal size. The larger (or major) groove is about 11.6 Å wide and about 8.5 Å deep, while the smaller (or minor) groove is about 6.0 Å wide and about 8.2 A deep. In A,T rich dsDNA regions, the minor groove is narrower, reportedly in the range of 3-4 Å. See Neidle, Nat. Prod. Rep., 2001, 18, 291-309. The compounds provided herein are crescent shaped, providing a conformational fit enabling them to nestle in the minor groove. Depending on molecular structure and the width of the minor groove, a compound may bind individually within the minor groove (the 1:1 mode), or it may bind side-by-side with another compound (the 2:1 mode). The binding may be sequence-selective, that is, the compound recognizes and selectively binds to particular DNA sequences. The binding sites identified herein—having a prevalence of A and T—have been selected for their identity with or similarity to promoter sequences of bacterial genes, which are typically A,T rich. The binding of the compound to the target site prevents formation of the complex necessary for the transcription of the corresponding bacterial gene, possibly by displacing or inhibiting the binding of essential transcription factors or enzymes, and results in the downregulation or shutting down of the gene. By downregulating multiple essential bacterial genes, the compounds herein are believed to ultimately cause bacterial death. Because multiple genes are affected, it is more difficult for bacteria to develop resistance. Additionally, antifungal activity can be predictive of cytotoxic effects in other eukaryotic cells. Accordingly, the present antibacterial compounds have reduced antifungal activity (as determined by activity against Candida albicans ATCC 38247).

### DESCRIPTION OF THE SPECIFIC EMBODIMENTS

Methods for Reducing Bacterial Gene Expression

In view of the more general discussion above, the present invention provides, in one aspect, methods for treating infection by Gram-positive bacteria in a mammal, by administering to the mammal an effective amount of a compound that binds noncovalently in the minor groove of duplex DNA, which compound:

i) binds with a dissociation constant of equal to or less than 100 nM to at least one of:

(a) a target sequence AAAAAGCAAAAA in the 351 base pair EcoRI/PvuII restriction fragment of a polynucleotide of SEQ ID NO. I;

(b) a target sequence AAAAAGACAAAAA in the 351 base pair EcoRI/PvuII restriction fragment of a polynucleotide of SEQ ID NO. I;

(c) a target sequence AAAAAGTACAAAAA in the 351 base pair EcoRI/PvuII restriction fragment of a polynucleotide of SEQ ID NO. I;

(d) a target sequence AGTACT in the 310 base pair EcoRI/PvuII restriction fragment of a polynucleotide of SEQ ID NO. II;

(e) a target sequence AATACT in the 310 base pair EcoRI/PvuII restriction fragment of a polynucleotide of SEQ ID NO. II;

(f) a target sequence ATTACT in the 310 base pair EcoRI/PvuII restriction fragment of a polynucleotide of SEQ ID NO. II;

(g) a target sequence TGACAATTAAT in the 352 base pair EcoRI/PvuII restriction fragment of a polynucleotide of SEQ ID NO. III;

(h) a target sequence GACAATTAATCA in the 352 base pair EcoRI/PvuII restriction fragment of a polynucleotide of SEQ ID NO. III;

(i) a target sequence AATTAATCAT in the 352 base pair EcoRI/PvuII restriction fragment of a polynucleotide of SEQ ID NO. III;

(j) a target sequence ACAATTA in the 352 base pair EcoRI/PvuII restriction fragment of a polynucleotide of SEQ ID NO. III; and

(k) a target sequence ACAATTAAT in the 352 base pair EcoRI/PvuII restriction fragment of a polynucleotide of SEQ ID NO. III;

ii) exhibits a MIC of less than or equal to 2 μg/mL against at least one of Enterococcus faecium ATCC 51559, Staphylococcus aureus ATCC 27660, Staphylococcus aureus ATCC 33591, Staphylococcus aureus ATCC 43300, and Streptococcus pneumoniae ATCC 51422;

iii) exhibits a MIC of greater than or equal to 32 μg/mL against Candida albicans ATCC 38247; and

iv) has a molecular weight of from 100 to about 1100.

Of particular note regarding the method above, is the identification of specific sequences that can be targeted for binding agents useful for reducing bacterial gene expression. A compound is deemed to bind to one or more of the recited target sequences if, when contacted with duplex DNA of SEQ. I.D. NO. I, II or III (with each complementary strand), the compound binds with the noted dissociation constant and exhibits at least 50%, more preferably 60%, 70%, 80% or even 90% overlap with the indicated residues. In the most preferred embodiments, the compound exhibits 100% overlap with the indicated residues. The sequence of DNA targeted by the compounds provided herein can be determined to nucleotide resolution using MPE Footprinting or alternatively using DNase and MPE footprinting to determine affinity and target sequence (see, Van Dyke, et al., Nucl. Acids Res. (1983) 11:5555 and Van Dyke, et al., Science (1984) 225:1122.

In therapeutic use for treating, or combatting bacterial infections in humans and other animals that have been diagnosed with bacterial infections, compounds having the functional properties described above, or advantageously having formula (I) below are administered. Typically administration of the compounds is in the form of pharmaceutical compositions thereof and such administration is made orally, parenterally and/or topically at a dosage to obtain and maintain a concentration, that is, an amount, or blood-level of active component in the animal undergoing treatment which will be antibacterially effective. Generally, such antibacterially effective amount of dosage of active component will be in the range of about 0.1 to about 100 mg/kg, more preferably about 3.0 to about 50 mg/kg of body weight/day. It is to be understood that the dosages may vary depending upon the requirements of the patient, the severity of the bacterial infection being treated, and the particular compound being used. Also, it is to be understood that the initial dosage administered may be increased beyond the above upper level in order to rapidly achieve the desired blood-level or the initial dosage may be smaller than the optimum and the daily dosage may be progressively increased during the course of treatment depending on the particular situation. If desired, the daily dose may also be divided into multiple doses for administration, e.g., two to four times per day.

The compounds of formula (I) according to this invention are administered parenterally, i.e., by injection, for example, by intravenous injection or by other parenteral routes of administration. Pharmaceutical compositions for parenteral administration will generally contain a pharmaceutically acceptable amount of the compound according to formula (I) as a soluble salt (acid addition salt or base salt) dissolved in a pharmaceutically acceptable liquid carrier such as, for example, water-for-injection and a suitably buffered isotonic solution, for example, having a pH of about 3.5-6. Suitable buffering agents include, for example, trisodium orthophosphate, sodium bicarbonate, sodium citrate, N-methylglucamine, L(+)-lysine and L(+)-arginine, to name a few. The compound according to formula (I) generally will be dissolved in the carrier in an amount sufficient to provide a pharmaceutically acceptable injectable concentration in the range of about 1 mg/mL to about 400 mg/mL. The resulting liquid pharmaceutical composition will be administered so as to obtain the above-mentioned antibacterially effective amount of dosage. The compounds of formula (I) according to this invention are advantageously administered orally in solid and liquid dosage forms.

In certain embodiments, the compounds are administered in combination with a second agent that is either an antibacterial or antimicrobial agent. Antibacterial agents useful in the present compositions and methods include in general the β-lactam antibiotics and the quinolone antibiotics. More particularly, the agents can be naficillin, oxacillin, penicillin, amoxacillin, ampicillin, cefotaxime, ceftriaxone, rifampin, minocycline, ciprofloxacin, norfloxacin, erythromycin, vancomycin, or an analog thereof. Antimicrobial agents useful in the present compositions and methods include in general sulfanilamide, sulfamethoxazole, sulfacetamide, sulfisoxazole, sulfadiazine, penicillins (e.g., penecillins G and V, methicillin, oxacillin, naficillin, ampicillin amoxacillin, carbenicillin, ticarcillin, mezlocillin and piperacillin), cephalosporins (e.g., cephalothin, cefaxolin, cephalexin, cefadroxil, cefamandole, cefoxitin, cefaclor, cefuroxine, loracarbef, cefonicid, cefotetan, ceforamide, cefotaxime, cefpodoxime proxetil, ceftizoxime, cefoperazone, ceftazidime and cefepime), aminoglycosides (e.g., gentamycin, tobramycin, amikacin, netilmicin, neomycin, kanamycin, streptomycin, and the like), tetracyclines (e.g., chlortetracycline, oxytetracycline, demeclocycline, methacycline, doxycycline and minocycline), macrolides (e.g., erythromycin, clarithromycin, azithromycin), and the like.

Pharmaceutical Compositions

The compounds described and provided herein, as well as those compounds identified using the criteria established above, can be prepared in a number of pharmaceutical compositions. In particular, the compounds can be prepared and administered in a wide variety of oral, topical and parenteral dosage forms. Thus, the compounds of the present invention can be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally. Accordingly, the present invention also provides pharmaceutical compositions comprising a pharmaceutically acceptable carrier or excipient and either a compound of formula (I) or a pharmaceutically acceptable salt of a compound of formula (I).

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired.

The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 1000 mg, preferably 1.0 mg to 100 mg according to the particular application and the potency of the active component. The composition can, if desired, also contain other compatible therapeutic agents.

Compounds for Inhibiting Bacterial Gene Expression

One class of compounds that can be used in the method of this invention has the following formula:

A-((L¹)ₚ-Ar¹)ₙ-L^{x}-B (I)

wherein A is a substituted or unsubstituted aryl or heteroaryl group, a substituted or unsubstituted heterocyclic group, an unsubstituted amino group or a mono- or di-alkyl amino group; the subscript n is an integer of from 2 to 7; the subscript p in each instance is an integer of from 0 to 1, indicating the presence or absence of each linking group (L¹); each L¹ is a linking group in which the superscript i is an integer of from 1 to n, and each linking group can be the same or different from the other linking groups and is selected from the group consisting of-NH—, —NR—, —CONH—, —SO₂NH—, —CONR—, —SO₂NR—, (C₁-C₆)alkylene, (C₁-C₆)heteroalkylene, and combinations thereof in which each R is independently (C₁-C₆)alkyl; Ar¹ is a substituted or unsubstituted aryl or heteroaryl group, in which the superscript i is an integer of from 1 to n and denotes the position away from A that is occupied by each aryl or heteroaryl group, and each Ar group can be the same or different from any other Ar group; L^{x} is a linking group selected from —NH—, —NR—, —CONH—, —SO₂NH—, CONR—, —SO₂NR—, (C₁-C₆)alkylene, (C₁-C₆)heteroalkylene, and combinations thereof; and B is a member selected from a substituted or unsubstituted aryl or heteroaryl group, a substituted or unsubstituted heterocyclic group, and an unsubstituted amino group or a mono- or di-alkyl amino group. The formula above is further meant to include all pharmaceutically acceptable salts, prodrug forms, protected forms and mixtures of isomers.

Thus, formula (I) includes, for example, polyaromatic compounds having the following formulae:

```
A—L¹—Ar¹—L²—Ar²—L³—Ar³—L⁴—Ar⁴—L⁵—Ar⁵—L^{X}—B
  A—L¹—Ar¹—L²—Ar²—Ar³—L⁴—Ar⁴—L⁵—Ar⁵—L^{X}—B
    A—L¹—Ar¹—L²—Ar²—L³—Ar³—L⁴—Ar⁴—L^{X}—B
      A—L¹—Ar¹—L²—Ar²—L³—Ar³—L^{X}—B
        A—L¹—Ar¹—L²—Ar²—L^{X}—B
          A—L¹—Ar¹—Ar²—L^{X}—B
```

A number of components are preferred for use in the methods of the present invention.

Turning first to the subscript n, in preferred embodiments the value of n is from 2 to 5. More preferably, n is 2, 3 or 4.

The first terminal group A can be, as noted above, a substituted or unsubstituted aryl or heteroaryl group, a substituted or unsubstituted heterocyclic group, or an amino group that is either an unsubstituted amino group or a mono- or di-alkyl amino group. In one group of embodiments, A is a substituted or unsubstituted aryl or heteroaryl group selected from phenyl, 1-naphthyl, 2-naphthyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 2-benzothienyl, 2-benzothiazolyl, purinyl, 2-benzimidazolyl, 2-indolyl, 1-isoquinolyl, 2-quinoxalinyl, 3-quinolyl, and 6-quinolyl. More preferably, A is a substituted or unsubstituted thienyl group, a substituted or unsubstituted phenyl group, a substituted or unsubstituted benzothienyl group, or a substituted or unsubstituted isoquinolinyl group. For those embodiments in which A is a substituted thienyl, phenyl, benzothienyl or isoquinolinyl group, the substituents are preferably selected from halogen, nitro, cyano, (C₁-C₆)alkyl, (C₁-C₆)alkoxy, (C₂-C₆)alkenyl, (C₂-C₆)alkynyl, halo(C₁-C₆)alkyl, halo(C₁-C₆)alkoxy, halo(C₂-C₆)alkenyl, and halo(C₂-C₆)alkynyl. More preferably, the substituents are selected from F, Cl, Br, nitro, cyano and halo(C₁-C₆)alkyl. Most preferably, the substituents are F, Cl, or Br. Particularly preferred A groups are 4,5-dibromo-2-thienyl, 3-chloro-2-thienyl, 3-fluoro-2-thienyl, 3-chloro-2-benzothienyl, 2-fluoro-4-chlorophenyl, 2,4-difluorophenyl, and isoquinolinyl.

The linking group components of formula I (e.g., L¹, L², L³ etc.) include —CONH—, —SO₂NH—, —CONR—, —SO₂NR—, (C₁-C₆)alkylene, —NH—, —NR—, (C₁-C₆)heteroalkylene, and combinations thereof wherein R is (C₁-C₆)alkyl, optionally substituted by one or more halogens. For the linking groups provided above, no particular orientation is implied. For example, the recitation —CONH— is meant to include —NHCO—. Additionally, the term “and combinations thereof” refers to a combination of components (e.g., 2, 3, or 4 components) that can be same or different, including for example, —CONH—(C₁-C₆)alkylene-CONH—, —(C₁-C₆)alkylene-CONH—, —(C₁-C₆)alkylene-CONH—(C₁-C₆)alkylene, —(C₁-C₆)alkylene SO₂NH—, and —CONR—(C₁-C₆)alkylene-SO₂NR—. Particularly preferred linking groups are —CONH—, —CONR— and —CONH—(C₁-C₆)alkylene-CONH—.

Turning next to the Ar components of the present antibacterial agents, each Ar can be the same or different and is preferably selected from substituted or unsubstituted forms of pyrrole, thiophene, thiazole, isothiazole, oxazole, isoxazole, pyrazole, pyrazine, pyridine, isoquinoline, benzothiazole, benzimidazole, benzoxazole, benzothiophene, and indole. Particularly preferred Ar components are selected from substituted or unsubstituted forms of pyrrole, thiophene, thiazole, isothiazole, oxazole, isoxazole, pyrazole, pyrazine, pyridine, benzothiophene, isoquinoline, pyridine and benzimidazole. When these Ar groups are substituted, the substituents are generally halogen or substituted or unsubstituted (C₁-C₆)alkyl groups. In one group of embodiments, the substituents are unsubstituted (C₁-C₆)alkyl groups, more preferably unsubstituted (C₁-C₄)alkyl, and most preferably, methyl or ethyl groups. In another group of embodiments, the substituents are substituted (C₁-C₆)alkyl groups in which the substituent on the alkyl group is a 5- or 6-membered unsubstituted heterocycle selected from piperidine, pyrrolidine, morpholine, piperazine, pyran and furan. Particularly preferred substituents on the Ar components are 2-(N-morpholinyl)ethyl, 2-(N-piperidinyl)ethyl and 2-(N-pyrrolidinyl)ethyl.

The symbol L^{x} represents yet another linking group component. In general, this linking group can be the same or different from any of the linking groups described above. In preferred embodiments, L^{x} is selected from —CONH—, —SO₂NH—, —CONR—, —SO₂NR—, (C₁-C₆)alkylene, (C₁-C₆)heteroalkylene, —CONH—(C₁-C₆)alkylene-, —CONH—(C₁-C₆)alkylene-CONH—, —CONH—(C₁-C₆)alkylene-CONH—(C₁-C₆)alkylene, —(C₁-C₆)alkylene-CONH—, —(C₁-C₆)alkylene-CONH—(C₁-C₆)alkylene, —(C₁-C₆)alkylene-SO₂NH—, and —CONH—(C₁-C₆)alkylene-SO₂NR—. In particularly preferred embodiments, L^{x} is selected from —CONH—, —CONR—, (C₁-C₆)alkylene, —CONH—(C₁-C₆)alkylene-, —CONH—(C₁-C₆)alkylene-CONH—, —CONH—(C₁-C₆)alkylene-CONH—(C₁-C₆)alkylene, —(C₁-C₆)alkylene-CONH—, and —(C₁-C₆)alkylene-CONH—(C₁-C₆)alkylene. In the most preferred embodiments, L^{x} is selected from CONH—, —CONH—(C₁-C₆)alkylene-, and —CONH—(C₁-C₆)alkylene-CONH—(C₁-C₆)alkylene.

The letter B in formula (I) represents a second terminal group that can be a substituted or unsubstituted aryl or heteroaryl group, a substituted or unsubstituted heterocyclic group, or an amino or mono- or di-alkyl amino group. The substituted or unsubstituted aryl or heteroaryl groups are preferably nitrogen-containing heteroaryl groups such as, for example, pyridine, thiazole, isothiazole, pyrrole, quinonline or isoquinoline. More preferably, the substituted or unsubstituted heteroaryl groups are pyridine, thiazole or isothiazole. Preferred substituents for the heteroaryl groups are unsubstituted (C₁-C₆)alkyl groups that are linear or branched. Similarly, the substituted or unsubstituted heterocyclic groups are nitrogen-containing heterocycles such as, for example, piperidine, morpholine, pyrrolidine, thiomorpholine and hexamethyleneimine (homopiperidine). Preferably, each of these heterocycles is unsubstituted other than the point of attachment to L^{x}.

In one group of particularly preferred embodiments, the compounds used in the present methods have the formula:

A-L¹-Ar¹-L²-Ar 2-L¹-Ar¹-L⁴-Ar¹-L^{x}-B (Ia)

In this group of particularly preferred embodiments, A is selected from substituted or unsubstituted thiophene, substituted or unsubstituted thiazole, and substituted or unsubstituted benzothiophene (thianaphthene). More preferably, A is a substituted or unsubstituted thiophene, still more preferably a substituted thiophene In the most preferred embodiments, A is a halogen-substituted thiophene. L¹ is preferably —CONH—, —CONR—, (C₁-C₆)alkylene, —CONH—(C₁-C₆)alkylene- or —CONR—(C₁-C₆)alkylene-. More preferably, L¹ is —CONH— or —CONR—, most preferably —CONH—. The first aryl group, Ar¹ is preferably a 5-membered heteroaryl moiety selected from pyrrole, thiazole, isothiazole and isoxazole. More preferably, Ar¹ is a substituted or unsubstituted pyrrole, wherein the substituents, when present are halogen or (C₁-C₄)alkyl. In the most preferred embodiments, Ar¹ is N-methyl pyrrole and the linking groups are attached at the 2- and 4-positions of the pyrrole ring. Continuing along formula (Ia), L² is preferably —CONH—, —CONR—, (C₁-C₆)alkylene, —CONH—(C₁-C₆)alkylene- or —CONR—(C₁-C₆)alkylene-. More preferably, L² is —CONH— or —CONR—, most preferably —CONH—. Preferred groups for each of Ar², Ar³ and Ar⁴ are the same as the preferred groups for Ar¹. L³ is preferably a linking group that combines amide and alkylene groups. Accordingly, L³ is preferably a linking group selected from —CONH—(C₁-C₆)alkylene, —CONH—(C₁-C₆)alkylene-CONH—, —CONH—(C₁-C₆)alkylene-CONH—(C₁-C₆)alkylene, —(C₁-C₆)alkylene-CONH— and —(C₁-C₆)alkylene-CONH—(C₁-C₆)alkylene. In each of the L³ linking groups, the alkylene portion is preferably methylene, ethylene, propylene or butylene, more preferably ethylene. In the most preferred embodiments of formula (Ia), L³ is —CONH—(C₂-C₄)alkylene-CONH—. Preferred embodiments of L⁴ in formula (Ia) are the same as those provided above for L². The linking group L¹, like L³ is preferably a combination of amide and alkylene groups. In particular, L^{x} is preferably —CONH—(C₁-C₆)alkylene, —CONH—(C₁-C₆)alkylene-CONH—, —CONH—(C₁-C₆)alkylene-CONH—(C₁-C₆)alkylene and —(C₁-C₆)alkylene-CONH—(C₁-C₆)alkylene. More preferably, L^{x} is —CONH—(C₁-C₆)alkylene-CONH—(C₁-C₆)alkylene. Still more preferably, L^{x} is —CONH—(C₁-C₃)alkylene-CONH—(C₂-C₅)alkylene. Within this group of preferred embodiments, the alkylene groups are preferably linear or branched, and optionally substituted with from 1 to 3 substituents that are halogen, methyl or ethyl.

The letter B represents the terminal functional group and is preferably a dialkyl amine or a nitrogen heterocycle (e.g., piperidine, hexamethyleneimine, morpholine, pyrrolidine, or thiomorpholine). For those preferred embodiments in which B is a dialkyl amine, most preferred are —NR¹R² in which R¹ and R² can be the same or different and individually have from one to four carbon atoms. For those preferred embodiments in which B represents a nitrogen heterocycle, an unsubstituted piperidine is most preferred.

Having provided preferred groups for each of the components of formula (Ia), one of skill in the art will appreciate that certain combinations of these preferred groups are particularly preferred. For example, in one groups of particularly preferred embodiments, A is a halogen-substituted thiophene (e.g., 4-bromothiophene or 4,5-dibromothiophene); Ar¹, Ar², Ar³ and Ar⁴ are each N-methylpyrrole with linking groups attached at the 2- and 4-positions; L¹, L² and L⁴ are each —CONH—; L³ is —CONH—(C₂-C₄)alkylene-CONH—; L^{x} is —CONH—(C₁-C₃)alkylene-CONH—(C₂-C₅)alkylene; and B is selected from dimethylamino, diethylamino, diisopropylamino, piperidine, pyrrolidine and hexamethyleneimine.

In another group of particularly preferred embodiments, the compounds used in the present methods have the formula:

A-L¹-Ar¹-L¹-Ar 2-L¹-Ar¹-L^{x}-B (Ib)

In this group of particularly preferred embodiments, A is selected from substituted or unsubstituted thiophene, substituted or unsubstituted benzene, substituted or unsubstituted isoquinoline, substituted or unsubstituted thiazole, substituted or unsubstituted benzothiophene (thianaphthene) and a substituted or unsubstituted 5- to 7-membered nitrogen heterocycle (e.g., piperidine, pyrrolidine, morpholine, hexamethyleneimine). More preferably, A is a substituted thiophene, substituted benzene, unsubstituted isoquinoline, substituted benzothiophene (thianaphthene) or a substituted or unsubstituted 6-membered nitrogen heterocycle (e.g., piperidine or morpholine). In this group of preferred embodiments for A, the substituents, when present, are preferably halogen, nitro, cyano, or (C₁-C₄)alkyl. Most preferably, the substituents are halogens selected from F, Cl and Br. L¹ is preferably —CONH—, —CONR—, (C₁-C₆)alkylene, —CONH—(C₁-C₆)alkylene-, —(C₁-C₆)alkylene-NH— or —NH—(C₁-C₆)alkylene-. More preferably, L¹ is —CONH—, —CONR—, or —(C₁-C₆)alkylene-NH—, most preferably —CONH— or —CH₂CH₂NH—. The remaining L groups (other than L^{x}) are all preferably —CONH— or —CONR—, most preferably —CONH—.

Turning next to the aryl groups in formula (Ib), the first aryl group, Ar¹ is preferably a 5-membered heteroaryl moiety selected from pyrrole, thiophene, thiazole, isothiazole and isoxazole. More preferably, Ar¹ is a substituted or unsubstituted pyrrole, substituted or unsubstituted thiophene, substituted or unsubstituted isoxazole, or a substituted or unsubstituted isothiazole, wherein the substituents, when present are halogen or (C₁-C₄)alkyl. In the most preferred embodiments, Ar is selected from pyrrole and N-methylpyrrole wherein the linking groups are attached at the 2- and 4-positions of the pyrrole ring; unsubstituted thiophene having the linking groups attached at the 2- and 4-positions; 4-chloroisothiazole having the linking groups attached at the 2- and 4-positions; and isoxazole having the linking groups attached at the 3- and 5-positions. Continuing along formula (Ib), preferred groups for each of Ar² and Ar³ are the same as the preferred groups for Ar¹. More preferably, each of Ar² and Ar³ are substituted pyrrole wherein the substituents are attached to the nitrogen atom and are selected from (C₁-C₄)alkyl and heterocyclyl(C₁-C₄)alkyl. Still more preferably, Ar² and Ar³ are selected from N-methylpyrrole, N-(2-(N-morpholino)ethyl)pyrrole. The linking group L^{x} is preferably an amide group or a combination of amide and alkylene groups. In particular, L^{x} is preferably —CONH—, —CONR——CONH—(C₁-C₆)alkylene and —CONH—(C₁-C₆)alkylene-CONH—. More preferably, L^{x} is —CONH— or —CONH—(C₁-C₆)alkylene. Still more preferably, L^{x} is —CONH— or —CONH—(C₁-C₃)alkylene. Within this group of preferred embodiments, the alkylene groups are preferably linear and unsubstituted.

The letter B represents the terminal functional group and is preferably a nitrogen heterocycle (e.g., piperidine, hexamethyleneimine, morpholine, pyrrolidine, or thiomorpholine) or a heteroaryl group selected from isothiazole and pyridine. For those preferred embodiments in which B is a nitrogen heterocycle, an unsubstituted piperidine, morpholine, thiomorpholine or hexamethyleneimine is most preferred.

Having provided preferred groups for each of the components of formula (Ib), one of skill in the art will appreciate that certain combinations of these preferred groups are particularly preferred. For example, in one groups of particularly preferred embodiments, A is a halogen-substituted thiophene (e.g., 3-chlorothiophene or 3-fluorothiophene), 3-chlorothianaphthene, 2-fluoro-4-chlorobenzene, piperidine, isoquinoline, or a 2,4-difluorobenzene; Ar¹, Ar² and Ar³ are each N-methylpyrrole, N-(2-(N-morpholino)ethyl)pyrrole or unsubstituted pyrrole with linking groups attached at the 2- and 4-positions, 4-chloroisothiazole, thiophene, isoxazole and isothiazole; L¹, L² and L³ are each —CONH—; L^{x} is —CONH—(C₁-C₃)alkylene-; and B is selected from morpholine, thiomorpholine, 3-methylisothiazole, pyridine, piperidine and hexamethyleneimine.

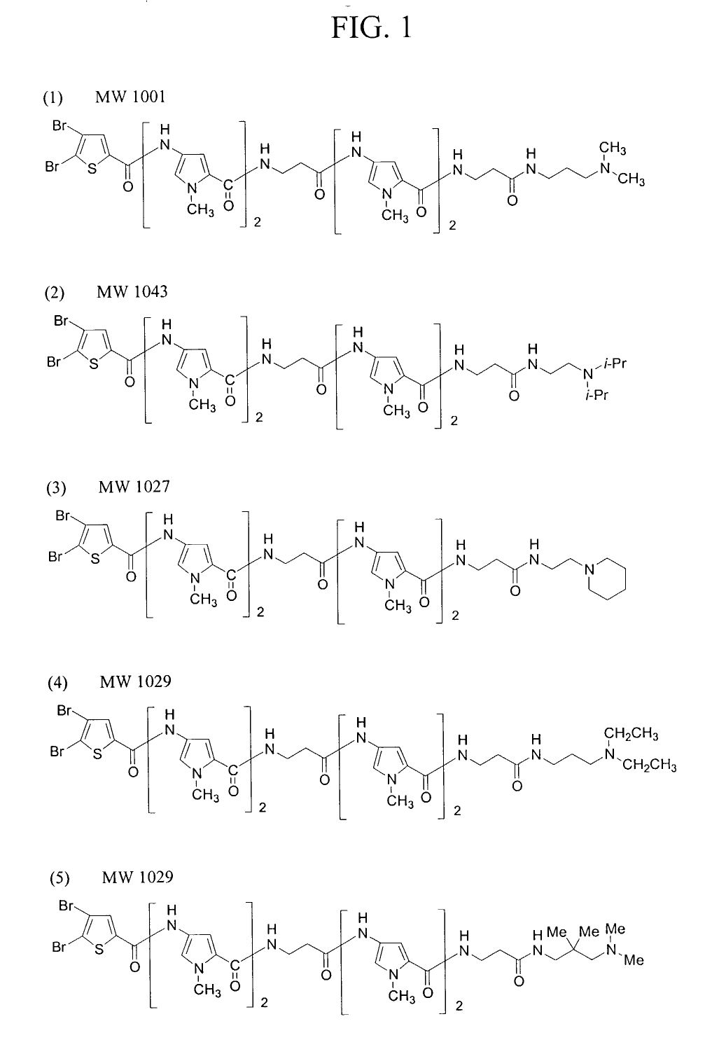

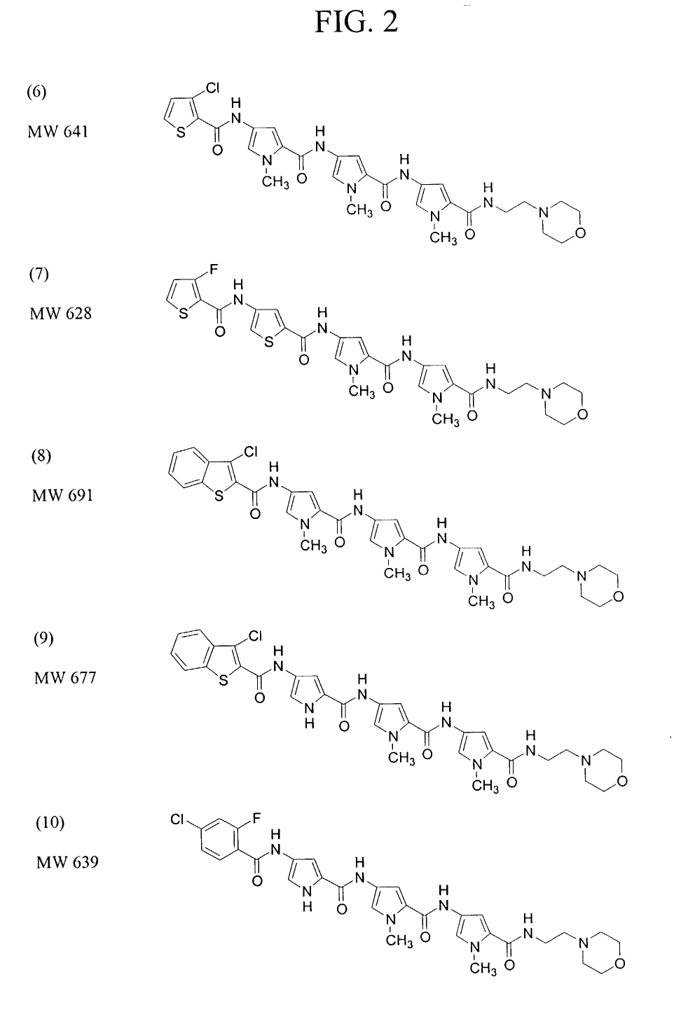

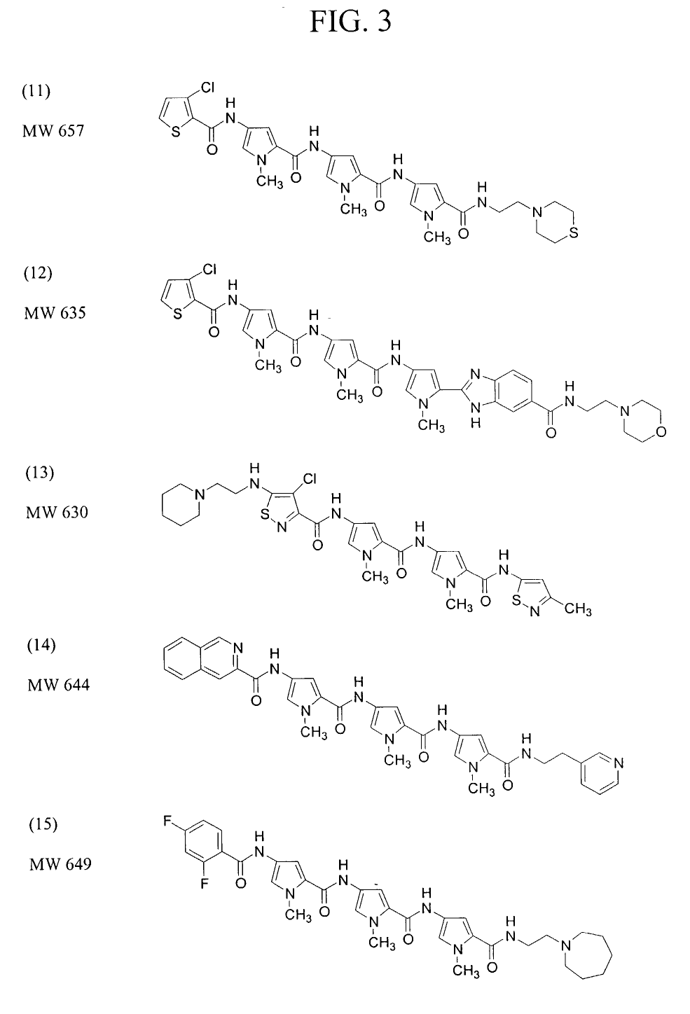

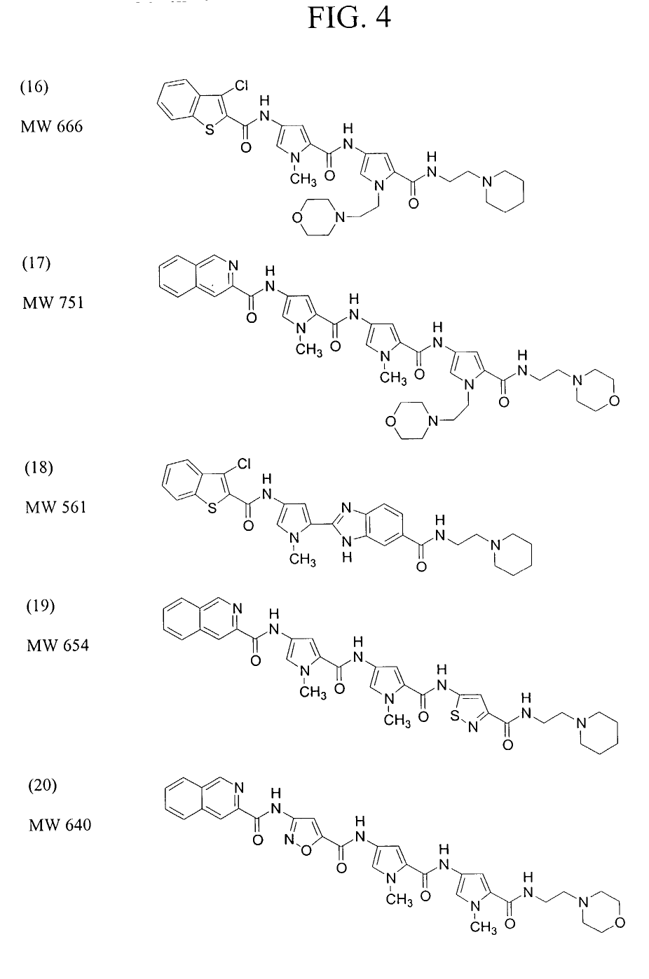

Illustrative specific compounds (1)-(20) are shown in FIGS. 1-4, along with their respective approximate molecular weights.

Certain compounds are further illustrated in the context of formula (I), below.

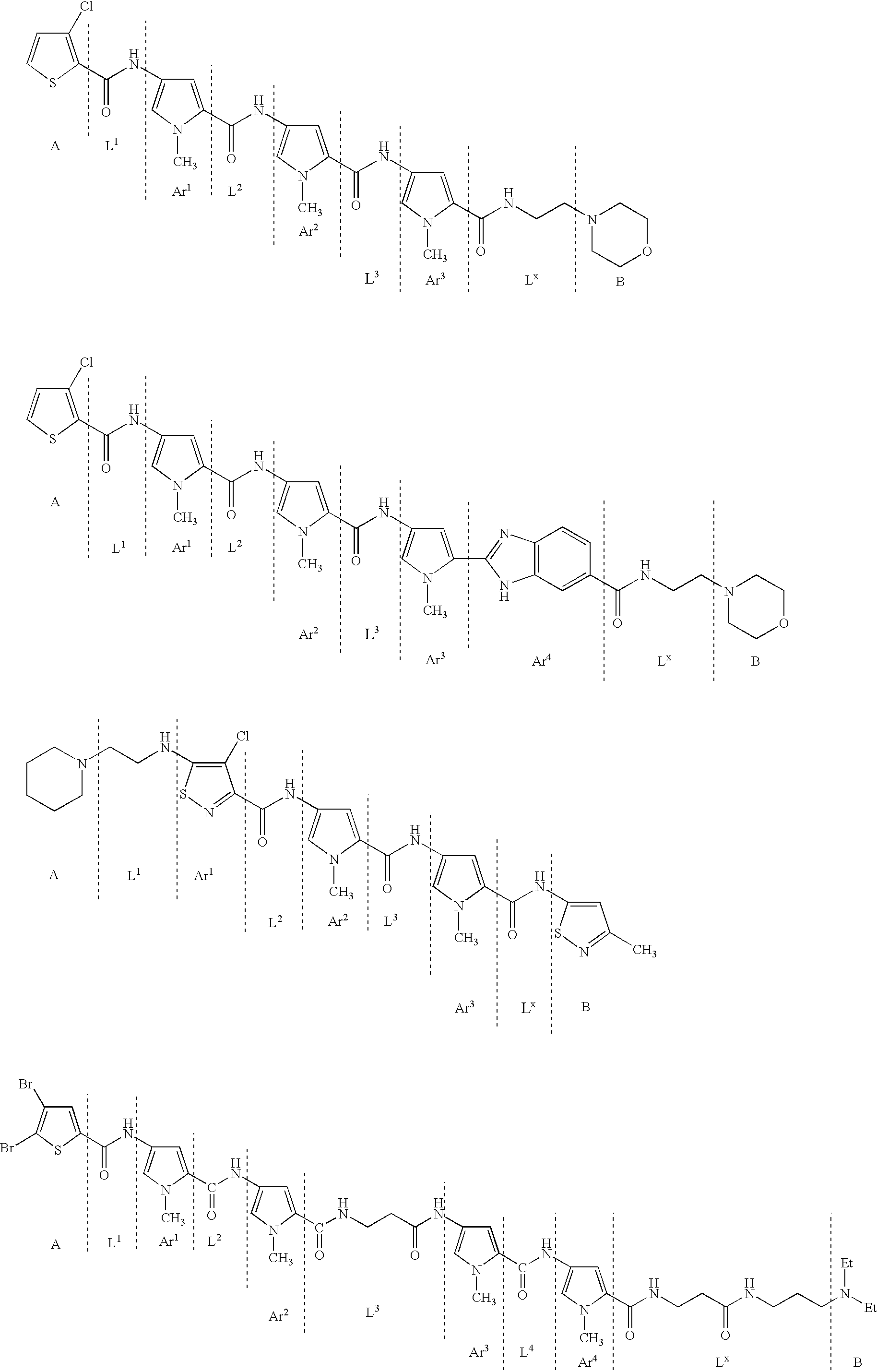

The practice of this invention can be further understood by reference to the following examples and procedures, which are provided by way of example and not limitations.

Synthesis of Compounds—General

Compounds having a halogenated thiophene-2-carboxylic acid residue, such as compounds 1-7 and 11-12, can be synthesized by the methods described in U.S. Provisional Application Ser. No. 60/286,454, filed Apr. 26, 2001, and entitled “Halogen-Substituted Thielnyl Compounds” (the “'454 application”), the disclosure of which is incorporated herein by reference.

Compounds having aryl-benzimidazole moieties, as in compounds 12 and 18, can synthesized by the methods described in U.S. Provisional Application Ser. No. 60/298,206, filed Jun. 13, 2001, and entitled “Aryl-Benzimidazole DNA-Binding Compounds” (the “'206 application”), the disclosure of which is incorporated herein by reference.

Compounds having isothiazole-3-carboxylic acid residues, as in compound 13, can be synthesized by the methods described in U.S. application Ser. No. 09/808,729, filed Mar. 14, 2001, and entitled “Charged Compounds Comprising a Nucleic Acid Binding Moiety and Uses Therefor” (the “'729 application”), the disclosure of which is incorporated herein by reference.

Additional documents disclosing synthetic techniques suitable for the synthesis of compounds of this invention include Dervan et al., U.S. Pat. No. 6,090,947 (2000) (the “'947 patent”); Kelly et al., Proc. Nat'l Acad. Sci. USA, July 1996, 93, 6981 (“Kelly”); and Wade et al., J. Am. Chem. Soc., 1992, 114, 8783 (“Wade”), the disclosures of which are incorporated herein by reference. Generally, the '947 patent relates to solid phase synthetic methods while Kelly and Wade relate to solution phase synthetic methods.

In the synthesis compounds incorporating one of the following groups (typically as a portion of A-L¹)

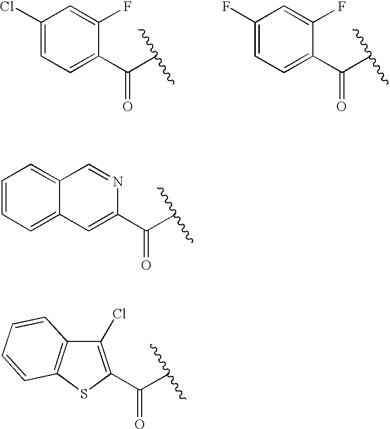

such as in compounds 8-10 and 14-20, the corresponding commercially available carboxylic acids were activated with 2-(1H-benzotriazole-1-yl)-1,1,3,3-tetramethyluronium hexafluorophosphate (“HBTU”, 0.95 equiv.) for 30 min at room temperature in N,N-dimethylformamide (“DMF”) or N-methylpyrrolidone (“NMP”) in the presence of diisopropylethylamine (“DIEA”).

In the synthesis of compounds having the B-terminal residue

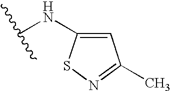

such as compound 13, commercially available 5-amino-3-methylisothiazole was used. It was to coupled to a desired carboxylic acid activated with O-(7-Azabenzotriazol-1-yl)-N,N,N′,N′-tetramethyluronium hexafluorophosphate (“HAPU”), which produces a more activated ester than HBTU, compensating for the lesser reactivity of the aromatic amine group compared to aliphatic amines. In some instances, the coupling reaction can be carried out at a more elevated temperature.

The use of HBTU and HATU activated esters to synthesize compounds of this invention generally followed the procedures described in the '947 patent, the '454, '206, and '729 applications and in Wade and Kelly.

Synthesis of Precursors of Pyrrole Carboxamide Residues Having Pendant Morpholine Groups.

The procedure for the synthesis of precursors for the introduction of pyrrole carboxamide precursors having pendant morpholine groups

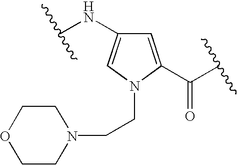

as exemplified by compounds 16 and 17, is summarized in Scheme A:

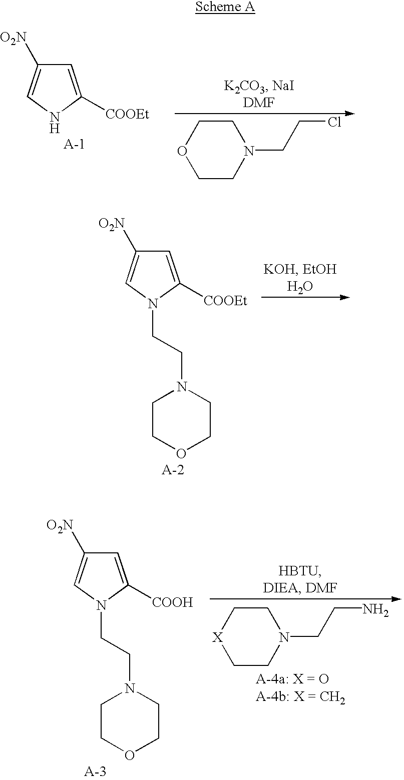

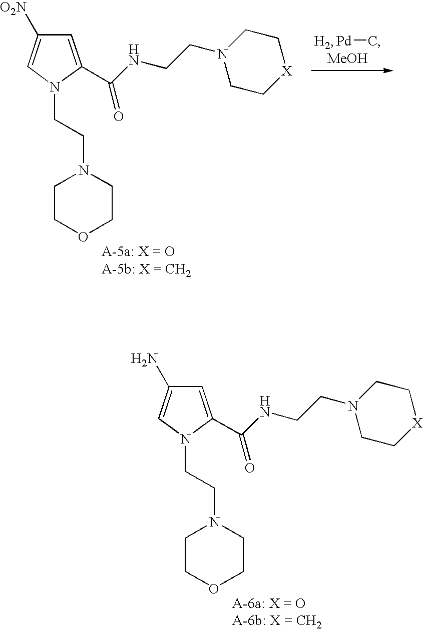

Synthesis of Ester A-2. A mixture of ethyl 4-nitropyrrole-2-carboxylate A-1 (20.00 g, 1.0 equiv.), 4-(2-chloroethyl)morpholine hydrochloride (28.28 g, 1.4 equiv.), NaI (16.28 g, 1.0 equiv.) and K₂CO₃ (28.78 g, 1.92 equiv.) in DMF (200 mL) was stirred at 60° C. for 10.5 h and poured into a mixture of H₂O and sat. aq. K₂CO₃ (550/50 mL). The resulting solution was extracted with AcOEt (4×, each 200 mL). The combined organic layers were dried (MgSO₄) and evaporated to give ester A-2 as pale yellow crystals (31.4 g, 97%). The ¹H-NMR spectrum was consistent with the assigned structure.

Synthesis of Acid A-3. A suspension of the ester A-2 (31.4 g, 1.0 equiv.) and K(OH (8.13 g, 2 equiv.) in EtOH (100 mL) and H₂O (100 mL) was stirred at room temperature (“RT”) for 16 h (complete dissolution occurred after 1 h). The mixture was acidified with 1M aq. HCl to pH 3.0 and the resulting precipitate collected by filtration and dried in vacuo to ive acid A-3 as a white solid (23.0 g, 81%). The ¹H-NMR spectrum was consistent with the assigned structure.

Synthesis of compounds A-5a and A-6a. A mixture of the acid A-3 (1.5 g, 1.0 equiv.) and HBTU (1.8 g, 1 equiv.) in DMF (8 mL) and DIEA (2 mL) was stirred at RT for 1 h, treated with the amine A-4a (0.70 mL, 1.1 equiv.) and stirred for 15 h at RT. The solution was added dropwise to ice-water (150 mL) and extracted with AcOEt (5×). The combined organic layers were dried (MgSO₄) and evaporated to give compound A-5a as a brown solid (1.6 g, ¹H-NMR spectrum consistent with the assigned structure). The crude product was dissolved in AcOEt (50 mL), treated with 10% Pd-C (ca. 100 mg), and stirred at RT under H₂ (1 atm) for 48 h. The mixture was filtered through Celite and the solids washed with MeOH. The filtrate was concentrated in vacuo, diluted with Et₂O (250 mL) and AcOEt (50 mL), and treated with HCl (g) for 1 min. Evaporation of the solvents gave compound A-6a as orange solids (1.7 g), which was subsequently used without further purification for the preparation of compounds such as compound 17, generally following the procedures described in the '454, '206, and '729 applications and in Wade and Kelly.

Synthesis of compounds A-5b and A-6b. A mixture of the acid A-3 (1.5 g, 1.0 equiv.) and HBTU (1.8 g, 1 equiv.) in DMF (8 mL) and DIEA (2 mL) was stirred at RT for 1 h, treated with the amine A-4b (0.75 mL, 1.1 equiv.) and stirred for 15 h at RT. The solution was added dropwise to ice-water (150 mL) and extracted with Et₂O (3×) and AcOEt (4×). The combined organic layers were dried (MgSO₄) and evaporated to give compound A-5b as a brown solid (1.7 g, ¹H-NMR spectrum consistent with the assigned structure). The crude product was dissolved in AcOEt (25 mL), treated with 10% Pd-C (ca. 50 mg), and stirred at RT under H₂ (1 atm) for 24 h. The mixture was filtered through Celite and the solids washed with MeOH. The filtrate was concentrated in vacuo, diluted with Et₂O (ca. 125 mL), and treated with HCl (g) for 1 min. The resulting precipitate was collected by filtration and dried in vacuo to give compound A-6b (1.78 g), which was subsequently used for the preparation of compounds such as compound 16 without further purification, generally following the procedures described in the '454, '206, and '729 applications and in Wade and Kelly.

Synthesis of Precursor for Isothiazole Carboxamide Residues.

The procedure for the synthesis of precursors for the introduction of isothiazole carboxamide residues

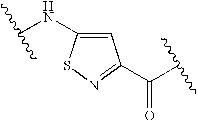

as exemplified by compound 19, is summarized in Scheme B:

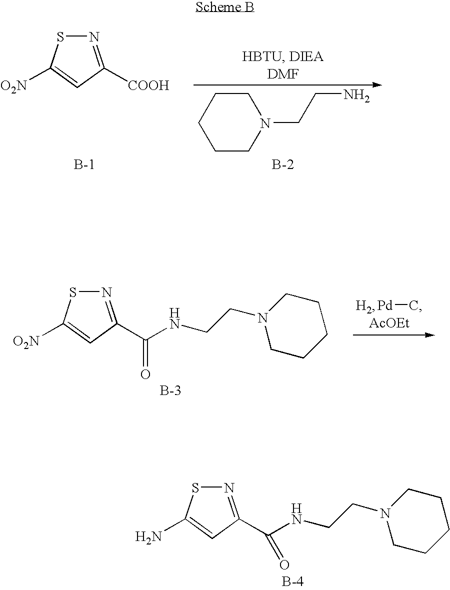

Synthesis of Compound B-3. A mixture of the nitro acid B-1 (300 mg, 1.2 equiv., J. Heindl, E. Schröder, H.-W. Kelm, Eur. J. Med. Chem.—Chimica Therapeutica, 1975, 10, 591-593, and references therein) and HBTU (620 mg, 1.14 equiv.) in DMF (2 mL) and DIEA (0.4 mL) was stirred at 37° C. for 18 h and treated with the amine B-2 (204 μL, 1.0 equiv.) and stirred for 24 h at RT. The mixture was diluted with H₂O (ca. 40 mL) and extracted with AcOEt (3×). The combined organic layers were dried (MgSO₄) and evaporated to give compound B-3 (400 mg, 98%). The ¹H-NMR spectrum was consistent with the assigned structure.

Synthesis of Compound B-4. A suspension of compound B-3 (400 mg, crude product from above procedure) and 10% Pd-C (ca. 500 mg) in AcOEt (30 mL) was stirred at RT for 24 h under H₂ (1 atm). The mixture was filtered through Celite and the solids washed with AcOEt. The filtrate was concentrated in vacuo, diluted with Et₂O, and treated with HCl (g) for ca. 1 min. The resulting precipitate was collected by filtration and dried to give compound B-4 as a light yellow powder (110 mg, 27%). The ¹H-NMR spectrum was consistent with the assigned structure (ca. 90 to 95% pure). Without further purification, this material was subsequently used for the synthesis of compounds such as compound 19, generally following the procedures described in the '454, '206, and '729 applications and in Wade and Kelly.

By way of further illustration, the synthesis of specific compounds for use in this invention is provided below. Those skilled in the art will appreciate that variant structures can be synthesized by reference to these syntheses, mutatis mutandis.

Synthesis of Compound (1)

Compound (1) was synthesized by the route shown in Scheme C. Part I of Scheme C describes the synthesis of the intermediate labeled Boc-Py-Py-OH, where “Py” denotes:

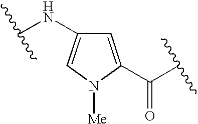

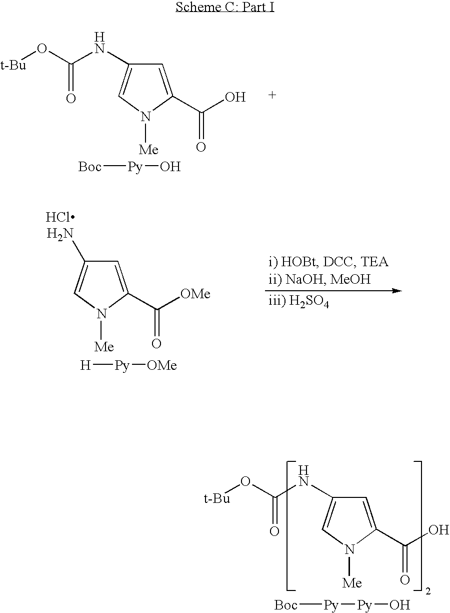

To a solution of Boc-Py-OH (40 g, 167 mmol) in 150 mL DMF was added 1.2 eq 20 hydroxybenzotriazole (“HOBt,” 27 g, 0.2 mmol) followed by 1.2 eq dicyclohexyl-carbodiimide (“DCC,” 40.4 g, 0.2 mmol). The solution was stirred for 5 hours at room temperature, after which the DCC was removed by filtration followed by a rinse with N,N-dimethylformamide (“DMF,” 50 mL). H-Py-OMe hydrochloride (34 g, 160 mmol) was added, followed by triethylamine (“TEA,” 80 mL) and the reaction was stirred at 50° C. for 10 hr. The reaction mixture was then added dropwise to a stirred solution of ice water (2 L) and the solution placed at 4° C. overnight. The resulting precipitate was collected by vacuum filtration and dried overnight to provide methyl 4-[t-butoxycarbonyl)amino]-1-methylpyrrole-2-(4-carboxamido)-1-methylpyrrole-2-carboxylate (53 g, 83% yield). The ester was dissolved in methanol (200 mL), NaOH (3M, 200 mL) was added, and the resulting mixture stirred for 3 hours at 50° C. Excess methanol was removed in vacuo and the resulting solution acidified to pH 3 using H₂SO₄. The resulting precipitate was collected by filtration and dried in vacuo to yield Boc-Py-Py-OH as a white powder (43 g, 90% yield).

Part II Scheme C describes the solid-phase synthesis of compound (1) proper. The starting point is commercially available Boc-β-alanine-PAM-resin (see also the '947 patent). This resin has a Boc-protected β-alanyl residue attached to the resin via a phenylacetamidomethyl (PAM) linkage:

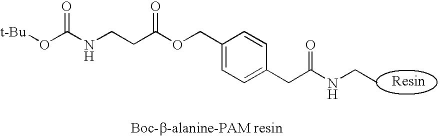

Hereinafter, the more compact notation

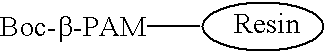

will be used for convenience, where “β” and “PAM” represent

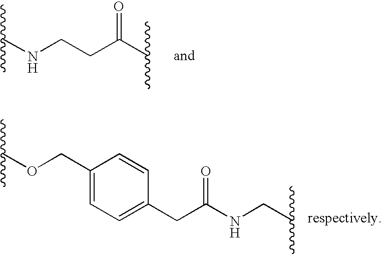

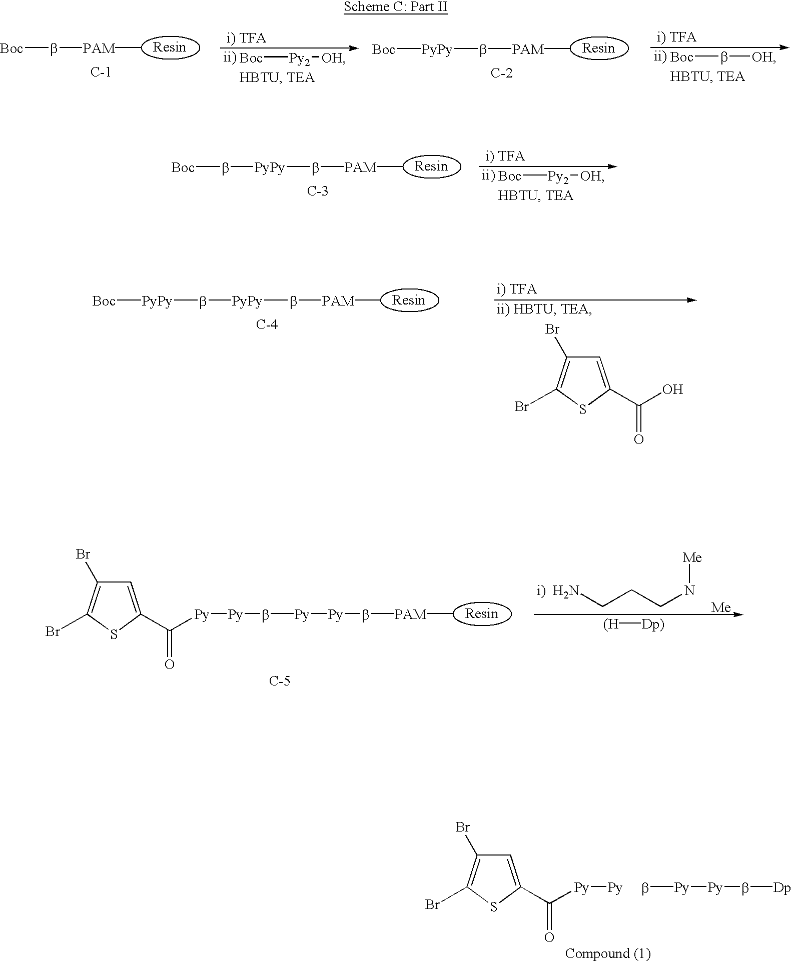

An Argonaut Quest 210 semi-automated synthesizer was used. This instrument has 20 disposable reaction vessels with volumes 10 of mL each, which can be washed, heated, mixed and drained using automated cycles or manually. Wash cycle A consists of three steps—step one is three cycles of adding NMP (5 mL) to each vessel, mixing for 2 minutes and draining the NMP from the vessels using a controlled flow of compressed nitrogen, steps two and three are the same as step one, but with the substitution of methanol and CH₂Cl₂, respectively, for NMP. Wash cycle B uses the same three steps as wash cycle one, using CH₂Cl₂, methanol and NMP in that order. The coupling cycle consists of heating the vessels to 37° C. and mixing for 2 hours. In the cleavage cycle, the vessels are heated to either 55° C. or 90° C. and mixed for 12 hours.

In the first cycle, Boc-β-alanine-PAM resin (C-1,200 mg) was placed in a vessel and manually washed with CH₂Cl₂. The protecting Boc group was then removed by manually adding 100% trifluoroacetic acid (“TFA,” 5 mL) and mixing for 20 minutes. The deprotected resin was washed using wash cycle B. Boc-Py-Py-OH (125 mg, 0.34 mmol) was then activated with HBTU (121 mg, 0.34 mmol) in NMP (0.66 mL) and TEA (0.33 mL) and added to the deprotected resin in a 2:1 solution NMP/TEA (1.0 mL). The Quest automated coupling step was used, followed by wash cycle A, to yield Boc-Py-Py-β-PAM resin (C-2).

In the second cycle, Boc-β-alanine-OH (65 mg, 0.34 mmol) was used instead of the Boc-Py-Py-OH, all other steps remaining the same, to form H-β-Py-Py-β-PAM resin (C-3). In the third cycle, the first cycle was repeated to form Boc-Py-Py-β-Py-Py-β-PAM resin (C-4). In the last cycle, the addition of 2,3-dibromothiophene-5-carboxylic acid to Boc-Py-Py-β-Py-Py-β-PAM resin, compound C-4 was deprotected and washed using wash cycle B. 2,3-Dibromothiophene-5-carboxylic acid (98.4 mg, 0.34 mmol) was then activated and added to the deprotected resin in a solution NMP/TEA (2:1, 1.0 mL). The automated coupling step was used, followed by wash cycle A and a manual wash with NMP to yield 2,3-dibromothiophene-5-Py-Py-β-Py-Py-β-PAM resin (C-5). The compound was cleaved from the resin by adding dimethylaminopropylamine (“H-Dp,” 3 mL) and using the automated cleavage cycle at 55° C. then purified by reversed phase preparative HPLC to yield compound (1), characterized by NMR.

Structurally related compounds (2)-(4) can be synthesized by an analogous method.

Synthesis of Compound (6)

Compound (6) was synthesized by the route shown in Scheme D, Parts I and II.

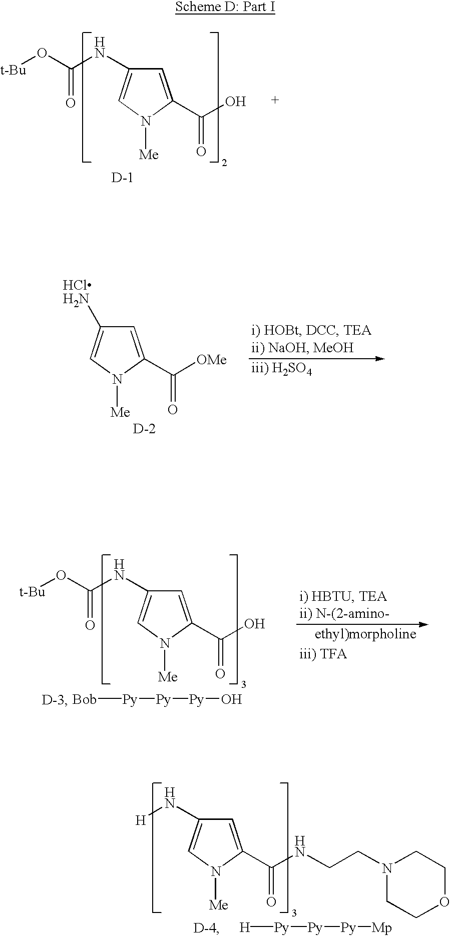

Part I relates to the synthesis of the intermediate Boc-Py-Py-Py-Mp (D-4).

To a solution of Boc-Py-Py-OH (D-1, 60.4 g, 167 mmol) in 150 mL DMF was added 1.2 eq HOBt (27 g, 0.2 mmol) followed by 1.2 eq DCC (40.4 g, 0.2 mmol). The solution was stirred for 5 hours at room temperature, after which the DCC was removed by filtration followed by a rinse with DMF (50 mL). H-Py-OMe hydrochloride (D-2, 34 g, 160 mmol) was added, followed by TEA (80 mL) and the reaction was stirred at 50° C. for 10 hours. The reaction mixture was then added dropwise to a stirred solution of ice water (2 L) and the solution stored at 4° C. overnight. The resulting precipitate was collected by vacuum filtration and dried overnight to provide methyl 4-[t-butoxycarbonyl)amino]-1-methylpyrrole-2-[4-carboxamido-1-methylpyrrole-2-(4-carboxamido-1-methylpyrrole)]-2-carboxylate. The ester was dissolved in methanol (200 mL), NaOH (3M, 200 mL) was added, and the resulting mixture stirred for 3 hours at 50° C. Excess methanol was removed in vacuo and the resulting solution acidified to pH 3 using H₂SO₄. The resulting precipitate was collected by filtration and dried in vacuo to yield Boc-Py-Py-Py-OH (D-3) as a white powder.

Boc-Py-Py-Py-OH (D-3, 0.1 mmol, 1 eq.) is activated with HBTU (0.095 mmole, 0.95 eq) in 50 mL DMF and 25 mL TEA for about 45 minutes at RT. N-(2-aminoethyl)-morpholine (0.12 mmol, 1.2 eq) is added to the mixture and the reaction is stirred at 37° C. overnight. The product mixture is concentrated in vacuo and TEA (150 mL) is added to the reaction, which is then stirred at room temperature for 3 hours. The solution is concentrated in vacuo, after which acetic acid (40 mL) and water (200 mL) is added. The solution is extracted with diethyl ether three times, then product D-4 is purified using reverse phase HPLC with a gradient of 1% acetonitrile/minute in 0.5% acetic acid.

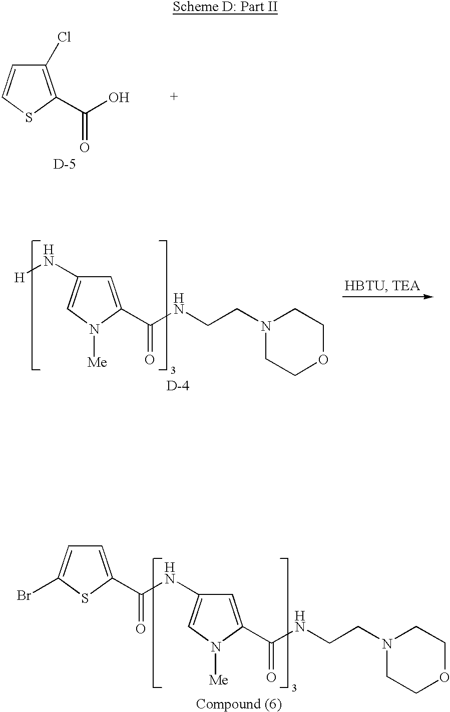

Part II of Scheme D describes the synthesis of compound (6) from the precursor made in Part I.

3-Chlorothiophene-2-carboxylic acid (D-5, 10 mmol, 1.2 eq.) is activated with HBTU (3.7 g., 9.8 mmol, 1.14 eq.) in DMF (20 mL) and TEA (13 mL). The solution is stirred for 10 minutes at RT, after which H-Py-Py-Py-Mp (D-4, 8.5 mmol, 1 eq.) is added. DMF (4 mL) is added to complete the transfer of the solid material, and the resulting solution is stirred at 37° C. overnight. The reaction is then dried in vacuo, 10% aqueous acetic acid (200 mL) is added and the product is purified using reversed phase preparative HPLC to yield compound (6).

Synthesis of Compound (7)

The synthesis of compound (7) is shown in Scheme E. Scheme E has three parts. In Part I, the synthesis of intermediate 4-boc(amino)-2-thiophene carboxylic acid (E-6) is described. In Part II, the synthesis of the intermediate 3-fluorothiophene-2-carboxylic acid (E-10) is described. Lastly, Part III describes the synthesis of compound (7) proper.

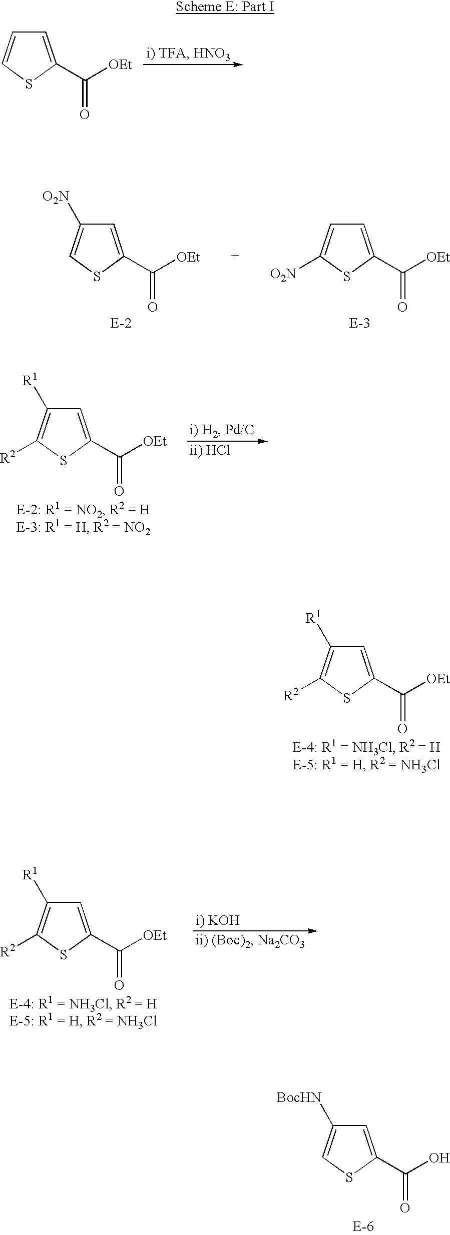

A solution of ethyl-2-thiophene carboxylate (E-1, 200 g, 1 mol) in TFA (200 mL) was slowly added to a mixture of TFA (900 mL) and fuming nitric acid (200 mL) at 5° C. The cooling was removed and the reaction mixture stirred at 45° C. for 14 hr, cooled to 10° C., and poured into vigorously stirred ice water (4 L). The resulting precipitate was collected by filtration and washed with ice water (2×). Lyophilization of the resulting solids gave a mixture of compounds E-2 and E-3 (2:3 ratio, by ¹H-NMR, 194.2 g, 79%).

A mixture of E-2 and E-3 (2:3 ratio, 20 g) in EtOAc (135 mL) and methanol (15 mL) was treated with 10% Pd-C (1 g) and stirred under H₂ (100 psi) for 6 days at room temperature. The reaction mixture was filtered through Celite and concentrated to a volume of 25 ml under reduced pressure. The residual solution was diluted with ethyl ether (500 mL), cooled to 0° C., and treated with HCl (gas) for 2 min. The resulting precipitate was collected by filtration and dried in vacuo to give a mixture of E-4 and E-5 (0.9:1 ratio by ¹H-NMR, 19.02 g, 93%).

A mixture of compounds E-4 and E-S (0.9/1, 15 g) in methanol (500 mL) was treated at 0° C. with a solution of KOH (9 g) in H₂O (75 mL) and stirred for 3 hi. The reaction mixture was then diluted with H₂O (400 mL) and washed with EtOAc (3×, 200 mL). The aqueous layer was neutralized to pH 6.5 with a 1 M aq. HCl solution, treated with Na₂CO₃ (15 g) and a solution of Boc anhydride (15 g) in dioxane (150 mL) and stirred for 24 h at RT. The reaction mixture was washed with EtOAc (3×, 200 mL), cooled to 0° C., acidified to pH 2.8 with aqueous HCl (50%), and extracted with EtOAc (3×, 200 mL). The combined organic layers were dried (MgSO₄) and evaporated. The remaining oil was dissolved in methanol and treated with activated carbon (5 g). The mixture was filtered through Celite and the filtrate evaporated to yield compound E-6.

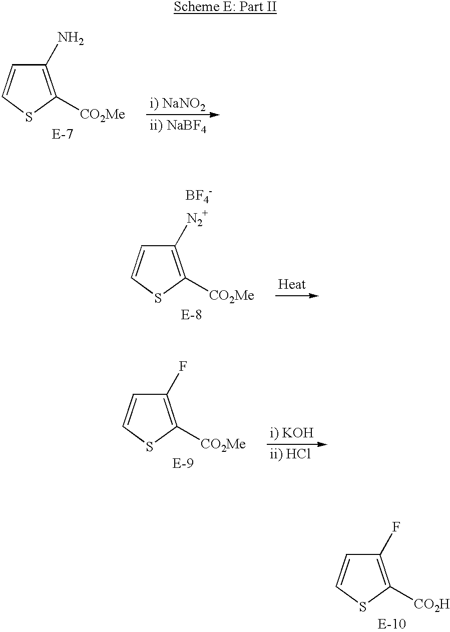

A suspension of aminothiophene ester E-7 (2 g, 12.72 mmol) in conc. aq. HCl (10 mL) and H₂O (20 mL) was treated dropwise with a solution of NaNO₂ (10.1 gram, 15.74 mmol) in H₂O (5 mL) at 0° C. The mixture was stirred at 0° C. for 20 minutes and treated with NaBF₄ (10 g) in H₂O (saturated). The solids were collected by filtration and washed with ice water, treated with tetrahydrofuran (“THF”), dried (MgSO₄), and evaporated.

The crude diazonium salt E-8 was distributed in a glass tube under a strong flow of N₂ that was passed through a cooling trap. The material was heated until the vigorous evolution of a gas that condensed upon cooling. The condensed material was collected with AcOEt. Evaporation gave fluorothiophene ester E-9 (720 mg) as a brown liquid.

A solution of the ester 3C (700 mg) and KOH (5 mL, 2 M) in ethanol (5 mL) was stirred for 28 hr at room temperature and diluted with H₂O (25 mL). The mixture was washed with AcOEt (2×) and acidified to pH 2 with 1M aq. HCl. Extraction of the mixture with AcOEt (2×), drying (MgSO₄) of the combined organic layers and evaporation gave 3-fluorothiophene-2-carboxylic acid E-10 (667 mg) as a brown solid.

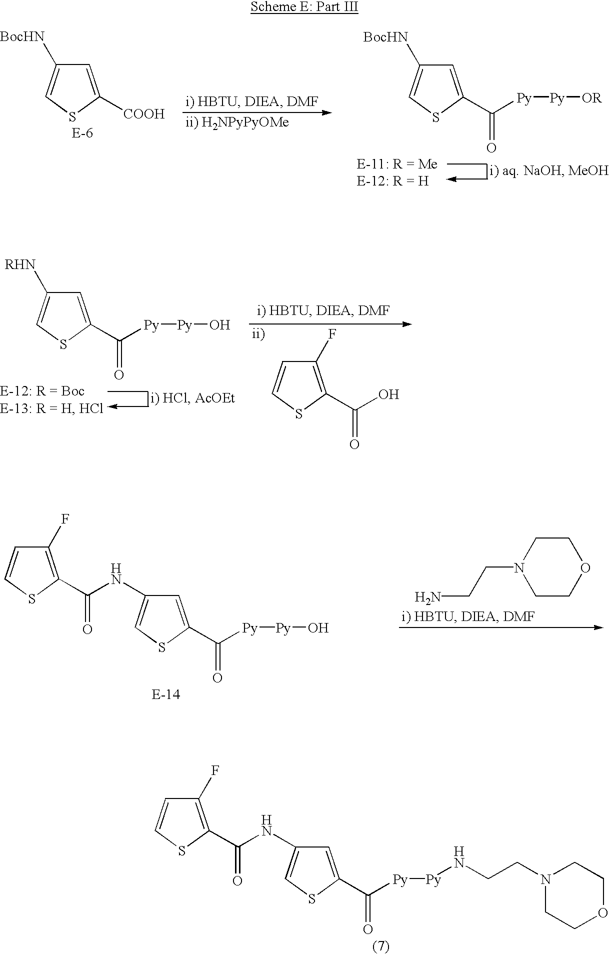

A solution of HOBt (1.51 g, 9.3 mmole, 1 eq.), DCC (2.31 g, 9.3 mmol, 1 eq.), and compound E-6 (2.2 g, 9.3 mmol, 1 eq.) in DMF (15 mL) was stirred for 45 min at room temperature, treated with H-Py-Py-OMe (2.9 g, 9.3 mmol, 1 eq., Bailly et al., J. Pharm. Sci. November 1989, 78, 11, 910-917) and diisopropylethylamine (“DIEA”) (2 mL), and stirred for 14 hr.

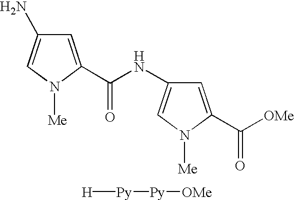

The mixture was added dropwise to ice-water (800 ml) and the resulting precipitate was collected by filtration and dried in vacuo to yield compound E-11 (4.2 g).

A solution of compound E-11(2.3 g) in MeOH (20 mL) was treated with a 2M solution of NaOH in H₂O (20 mL) and stirred for 3 hours at 50° C. The reaction mixture was diluted with H₂O and acidified to pH 2 with aqueous 1M HCl and extracted (AcOEt) three times. The combined organic layers were dried (MgSO₄) and evaporated to give acid E-12 (1.83 g, 83%).

Acid E-12 (0.70 g) was treated with a solution of ethyl acetate (saturated with HCl, 10 ml) and stirred at 4° C. for 30 minutes. The suspension was then added dropwise into ethyl ether (400 mL), from which the solid was filtered and dried in vacuo to yield amino acid E-13 (357 nmg).

A solution of 3-fluorothiophene-2-carboxylic acid (0.4 mmol, 1 eq.), HBTU (156 mg, 0.4 mmole, 1 eq.) in NMP (1 mL) and DIEA (0.1 mL) is stirred for 45 minutes at 37° C., treated with the amino acid E-13 (140 mg, 0.4 mmol, 1 eq.), and stirred for 12 hr. The reaction mixture is added dropwise to ice water (400 mL) to form a precipitate, which is then filtered and dried in vacuo. The product compound E-14 (0.08 mmol) is then treated with HBTU (40 mg, 0.1 mmol) in NMP (0.5 mL) and DIEA (0.05 mL) for 2 hours at room temperature, after which 4-(2-aminoethyl)morpholine (1.1 mL) is added and allowed to react for 15 hours at RT. The mixture is diluted with AcOH/H₂O, and washed with ethyl ether (3×).

HPLC purification of the aqueous phase gives compound (7).

Synthesis of Compound (12)

The synthesis of compound (12) is shown in Scheme F:

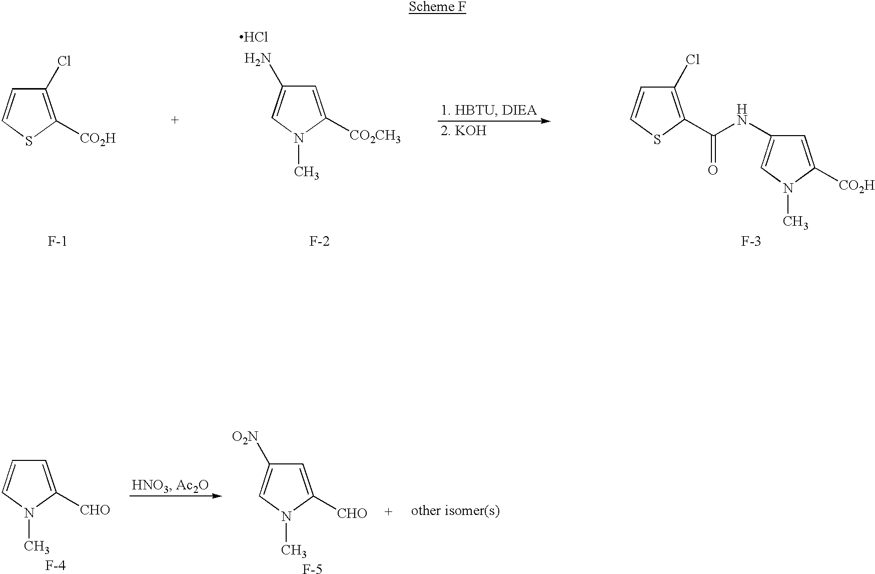

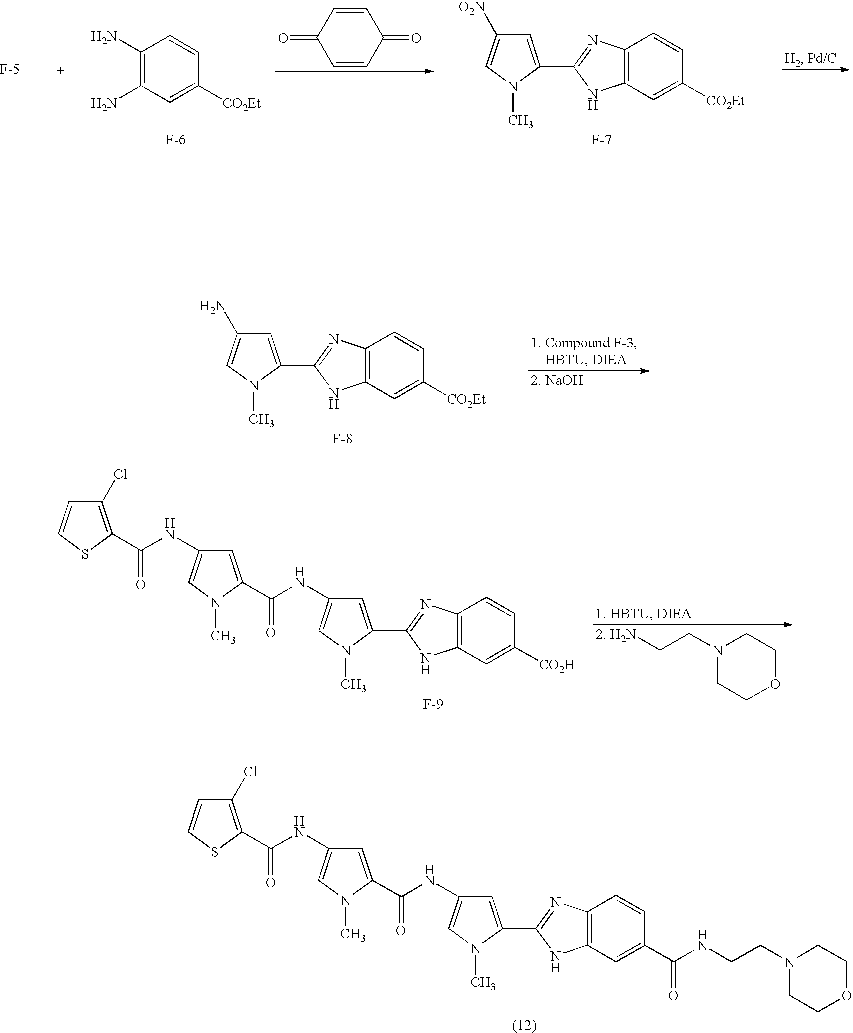

Synthesis of intermediate carboxylic acid F-3. To 3-chlorothiophene-2-carboxylic acid (F-1, 5.31 g, 32.6 mmol, commercially available) in DMF (30 mL) was added HBTU (11.8 g, 31.1 mmol) and diisopropylethylamine (“DIEA,” 6 mL). The reaction was stirred at room temperature for 30 min. To the solution was added methyl 4-amino-1-methylpyrrole-2-carboxylate hydrochloride (F-2, 5.19 g, 27.2 mmol). The reaction was stirred at room temperature for 12 h. The reaction was poured dropwise into stirring ice water (800 mL). The precipitate was collected over a coarse frit, washed with hot water, and lyophilized to provide 9.1 g (112% yield) of crude methyl ester of compound F-3. To the methyl ester (8.1 g) was added water (50 mL), ethanol (50 mL), and KOH (5 g). The reaction was stirred at room temperature for 12 h. To the reaction was added water (500 mL). The solution was extracted with ethyl acetate (1×100 mL). The aqueous layer was cooled to 0° C. and acidified to pH 2 with 7 M HCl. The resulting precipitate was filtered and washed successively with water, then dried in vacuo to provide near quantitative yield of the intermediate carboxylic acid F-3.

Nitration of N-methylpyrrole carboxaldehyde F-4. To acetic anhydride (240 mL) cooled at 0° C. was added fuming nitric acid (33.5 mL). After cooling, the mixture was added dropwise via addition funnel to a solution of N-methylpyrrole carboxaldehyde (F-4, 50 g, 458 mmol, commercially available) in acetic anhydride (240 mL) cooled to −40° C. in dry ice acetonitrile bath. Following addition of the nitration reagent and upon temperature stabilization, the reaction was allowed to slowly reach 10° C. At this point the temperature can increase rapidly. Between 10 and 20° C., the solution was immediately poured onto ice (480 g). The reaction was allowed to sit at room temperature overnight. Crystals formed. The solution was brought to 5° C. for 1 h, then filtered. The crystals formed were recrystalized from 100 mL of ethanol to provide 10.1 g of the desired isomer F-5. Further crystallization from ethanol provided an additional 7.64 g of desired isomer F-5.

Synthesis of benzimidazole intermediate F-8. A solution of 3-nitro pyrrole-5-carboxaldehyde (F-5, 10 g, 64.9 mmol) and ethyl-3,4-diaminobenzoate (F-6, 12.4 g, 69.1 mmol) in DMF (325 mL) was brought to 80° C. and stirred for 1 h. To the reaction was added benzoquinone (10.6 g, 97.6 mmol). The reaction was brought to 120° C. and stirred for 2 h. Solvent was removed in vacuo and 600 mL of dichloromethane were added to the solids. The suspension was boiled down to half volume, then stored at −25° C. for 1 h. Solids were filtered, rinsed with dichloromethane until filtrate ran clear. The solids were brought up in ethanolic HCl. Solvent was removed in vacuo. The product was crushed, precipitated from ethanol (600 mL), and filtered, and rinsed with cold ethanol (100 mL). Removal of ethanol from solids provided 17.49 g of the HCl salt of the nitro analog of compound F-8. To the HCl salt (0.37 g, 1.17 mmol) was added DMF (6 mL) and 10% Pd on carbon (0.3 g). The flask was fitted with a H₂ balloon and stirred overnight. The solution was filtered to provide compound F-8, which was used in subsequent steps without removal of the solvent.

Coupling of compounds F-3 and F-8. In a separate flask, compound F-3 (0.122 g, 0.43 mmol) was activated with HBTU (0.16 g, 0.41 mmol), DIEA (0.10 mL, 0.59 mmol), in DMF (1 mL). A solution of compound F-8 representing a theoretical amount of 0.39 mmol was added to activated compound F-3. The reaction was shaken in a 37° C. incubator for 2 h. Solvents were removed in vacuo. The resulting crude ethyl ester of compound F-9 was suspended in MeOH (3.2 mL) and 2 N NaOH (3.2 mL). The reaction was stirred at 60° C. overnight. MeOH was removed in vacuo. The basic solution was neutralized with 2 N HCl. The precipitated compound F-9 was filtered and washed with water. Excess water was removed in vacuo.

Conversion to compound (12). The resulting crude compound F-9 (0.17 g, 0.32 mmol) was activated with HBTU (0.12 g, 0.32 mmol), DIEA (0.11 mL, 0.65 mmol), in DMF (1 mL). To the activated compound F-9 was added N-aminoethylmorpholine (0.21 mL, 1.6 mmol). The solution was shaken at 37° C. for 2 h. Solvents were removed in vacuo. The final product was purified as before by reverse phase HPLC to yield 67 mg of compound (12). ¹H NMR δ_{H} (DMSO-d₆) 10.2 (s, 1H), 10.1 (s, 1H), 9.57 (s, 1H), 8.69 (s, 1H) 7.87 (d, 1H, J=5.6 Hz), 7.74 (d, 1H, J=8.0 Hz), 7.36 (s, 1H), 7.30 (s, 1H), 7.19 (d, 1H, J=5.2 Hz), 7.14 (s, 1H), 7.10 (s, 1H), 4.08 (s, 3H), 4.02 (m, 2H), 3.89 (s, 3H), 3.62 (m, 7H), 3.18 (m, 4H) m/z (ES) 636.1 (MH⁺).

Synthesis of Compound (20)

Compound (20) was synthesized according to Scheme G.

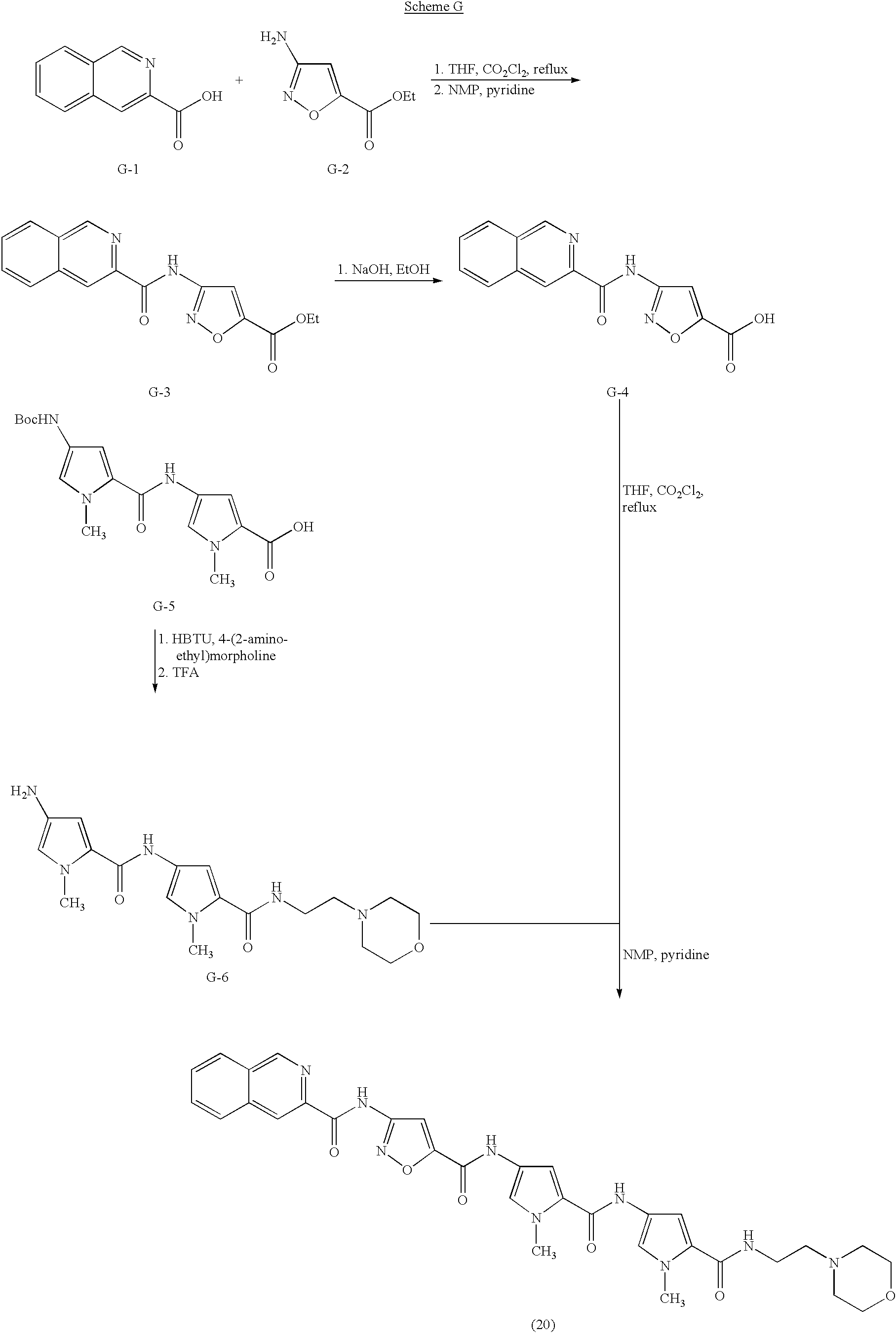

Oxalyl chloride (1.67 mL, 19.19 mmol) was added drop-wise to a suspension of isoquinoline-3-carboxylic acid (G-1, 332.3 mg, 1.92 mmol) in THF (2 mL) and the reaction heated at reflux (oil bath 85° C.) for 3 hours. All volatile components were then removed in vacuo. The resulting solid (presumed acid chloride) was then dissolved in NMP (1 mL) and pyridine (1 mL), and ethyl 3-aminoisoxazole-5-carboxylate G-2 (prepared as described in Lepage et al., FR 2,750,425 (1998), 300 mg, 1.92 mmol) was then added. The reaction was stirred at room temperature for 3 hours. The mixture was then added drop-wise to a rapidly stirred solution of ice-cold water (2 mL), which caused precipitation of the desired product ester-amide G-3 as a white solid (300 mg, 50%). This was filtered and dried by lyophilisation. The ¹H-NMR spectrum was consistent with the assigned structure.

Sodium hydroxide (1N, 2 mL, 2 mmol) was added to a solution of ester-amide G-3 (250 mg, 0.803 mmol) in ethanol (2 mL). The reaction was stirred for 30 minutes at room temperature, at which point, TLC analysis indicated complete consumption of the starting material. The mixture was then acidified to pH 2-3 with a 2N solution of hydrochloric acid, which caused precipitation of the product acid G-4 as a white solid (245 mg, quantitative yield). This was filtered and dried by lyophilisation. The 1H-NMR spectrum was consistent with the assigned structure.

Carboxylic acid G-5, having a Boc-protected amino group, was converted to amide-amine G-6 as follows: Compound G-5 was activated with HBTU (0.95 eq.) in DMF/TEA at RT for 45 min, followed by addition of 4-(2-aminoethyl)morpholine (1.2 eq.) and reaction at 37° C. overnight. Volatiles were removed in vacuo, and TFA was added. The reaction mixture was stirred at RT for 3 hr. Work-up yielded compound G-6. See the '454 application for the synthesis of compound G-5 and analogous reactions thereof.

Oxalyl chloride (0.22 mL, 2.54 mmol) was added drop-wise to a suspension of acid G-4 (72 mg, 0.254 mmol) in THF (1 mL) and the reaction heated at reflux (oil bath 85° C.) for 3 hours. All volatile components were then removed in vacuo. The resulting solid (presumed acid chloride) was then dissolved in NMP (0.5 mL) and pyridine (0.5 mL). A solution of amine G-6 (105 mg, 0.254 mmol) in NMP (1 mL) and DIEA (0.5 mL) was then added and the reaction stirred at 60° C. for 12 hours. The reaction mixture was then diluted with 50% acetic acid solution and directly purified by HPLC to give the desired product, compound (20) (25 mg, 16%). The ¹H-NMR spectrum was consistent with the assigned structure.

Anti-bacterial Activity

In vitro antibacterial activity data were collected for the following Gram-positive bacteria: Staphylococcus aureus (ATCC 27660, ATCC 33591 and ATCC 43300, all methicillin resistant strains (MRSA's)); Streptococcus pneumoniae (ATCC 51422, a penicillin resistant strain (PRSP)), Enterococcus faecium (ATCC 51559, and/or a vancomycin resistant strain (VRE)). Additionally, antifungal activity data were collected for Candida albicans (ATCC 38247). Minimal inhibition concentrations (MIC's) were determined using the National Committee for Clinical Laboratory Standards (NCCLS) broth microdilution assay in microtiter plates, as set forth in: (1) the guidelines of the National Committee for Clinical Laboratory Standards (NCCLS) Document M7-A4 (NCCLS, 1997); (2) the guidelines of the National Committee for Clinical Laboratory Standards (NCCLS) Document M11-A4 (NCCLS, 1997); and (3) the guidelines and reference method of the National Committee for Clinical Laboratory Standards (NCCLS) Document M27-T (NCCLS, 1995). For antifungal essays, the method recommended in Murray, PR., 1995 Manual of Clinical Microbiology (ASM Press, Washington, D.C.), was employed. The results are presented in Table II below.

TABLE II
Biological Activity

| | Organism (Minimum Inhibitory | | | | | |
|---|---|---|---|---|---|---|
| | Concentration (MIC), μg/mL) | | | | | |
| Compound | A | B | C | D | E | F |
| 1 | ++ | + | ND | − | ++ | >32 |
| 2 | ++ | − | ND | + | ++ | >32 |
| 3 | ++ | ++ | ND | + | ++ | >32 |
| 4 | ++ | ++ | ND | − | ++ | >32 |
| 5 | ++ | ++ | ND | − | ++ | >32 |
| 6 | +/− | + | − | ++ | ND | >32 |
| 7 | + | + | + | + | ++ | >32 |
| 8 | ++ | ++ | ++ | ++ | ++ | >32 |
| 9 | ++ | ++ | + | + | ++ | >32 |
| 10 | + | − | ND | + | − | 8-32 |
| 11 | + | − | ND | + | ++ | >32 |
| 12 | ++ | − | ND | ++ | ++ | >32 |
| 13 | ++ | ND | ND | ND | ND | >32 |
| 14 | ++ | − | ND | − | + | >32 |
| 15 | ++ | − | ND | + | ++ | 8 |
| 16 | ++ | ++ | ND | ++ | ++ | 4 |
| 17 | ++ | − | ND | ++ | ++ | >32 |
| 18 | ++ | ++ | ND | ++ | ++ | >32 |
| 19 | ++ | + | ND | ++ | ++ | >32 |
| 20 | ++ | ND | ND | ND | ND | >32 |

Key:
Col. A = S. aureus ATCC 27660
Col. B = S. aureus ATCC 33591
Col. C = S. aureus ATCC 43300
Col. D = E. faeciuin ATCC 51559
Col. E = S. pneunloniae ATCC 51422
Col. F = C. albicans ATCC 38247
+ = MIC ≦ 2
++ = MIC ≦ 1
− = MIC > 2
+/31 = Inconsistent data points
ND = No Data

Murine Neutropenic Thigh Model

This example demonstrates in vivo efficacy against infection by methicillin resistant Staphylococcus aureus ATCC 33591, using a murine neutropenic thigh model.

A S. aureus ATCC 33591 culture was grown to log phase overnight and diluted in phosphate buffered saline (pH 7.2) to an optical density of about 0.1 at 600 nm, giving an approximate concentration of 10⁸ cfu/mL. The suspension was diluted 1:100 in phosphate buffered saline (pH 7.2) for a final concentration of 10⁶ cfu/mL.

Outbred female CFI mice (approx. 20 gram body weight) were rendered netutropenic by treatment with cyclophosphamide (200 mg/kg body weight, intraperitoneal injection) at 2 and 4 days prior to inoculation. Groups of 5 mice were inoculated with 0.05 mL of the bacteria (approx. 10⁶ cfu/mL) into the anterior thigh. Each group was treated intravenously two hours post infection with vehicle (phosphate buffered saline) or test compound. The mice were sacrificed at either 6 or 24 hrs after treatment and thighs were collected aseptically. Each thigh was weighed, placed into sterile saline, and homogenized. The tissue homogenates were diluted appropriately for plating on agar plates. Colony counts were recorded (cfu/gram) and compared to control groups. The data are presented in Table III below:

TABLE III
Murine Neutropenic Thigh Model

| | | |
|---|---|---|
| | | Colony Count |
| Compound No. | Dose | (log cfu/gram) |

| | | | |
|---|---|---|---|
| (Time) | (mg/kg) | Compound | Vehicle |
| 9 (6 hr) | 80 | 6.17 | 7.83 |
| 12 (24 hr) | 50 | 6.96 | 8.03 |

In vivo efficacy was shown by a decrease in colony count (log cfu/gram of tissue) in the compound-treated animals when compared against the colony count in animals given only the vehicle.

Mouse Protection Assay

This example demonstrates in vivo efficacy against infection by methicillin resistant Staphylococcus aureus ATCC 33591, using a mouse protection assay.

A S. aureus ATCC 33591 culture was grown to log phase overnight and diluted in phosphate buffered saline (pH 7.2) to an optical density of about 0.1 at 600 nm, giving an approximate concentration of 10⁸ cfu/mL. Porcine mucin was added to the suspension to a final concentration of 5% mucin. The suspension was diluted 1:100 for a final concentration of 10⁶ cfu/mL.

Female balb/c mice (20 g body weight) were injected intraperitoneally with 0.5 mL of bactenal suspension (10⁶ cfu/mL). Vehicle (phosphate buffered saline, pH 7.2) or test compound were administered intravenously at 2, 8, 18, and 24 hours post infection. The animals were monitored twice daily and survival counts were recorded up to 48 hours post infection. The results are provided in Table IV:

TABLE IV
Murine Protection Assay

Survival at 48 hrs (%)

| Compound No. | Dose (mg/kg) | Compound | Vehicle |
|---|---|---|---|
| 6 | 50 | 86 | 14 |

In vivo efficacy was shown by an increase in survival at 48 hr post infection in the compound-treated animals, compared to vehicle treated animals.

DNA Binding

Compounds (1) through (20) according to this invention were screened for their ability to bind to specified DNA target sequences, using DNase I footprinting. Generally, the procedure described in Dervan, WO 98/50582 (1998), was followed.

Three double stranded circular plasmids A, B, and C were used to prepare double stranded DNA-binding probes containing the target sequences for the DNase I footprint titration experiments. Plasmids A, B, and C had nucleotide sequences given by SEQ ID NO. I, SEQ ID NO. II, and SEQ ID NO. III, respectively.

Plasmid A was prepared by hybridizing two sets of 5′-phosphorylated complementary oligonucleotides, the first set being

the first set being
5′-CCGGGAACGTAGCGTACCGGTGCAAAAAGCAAAAACGCTCGACGCCG
CAAAAAGACAAAAAGGCTCGA-3′
and
5′-GGCGTCGAGCCTTTTTGTCTTTTTGCGGCGTCGAGCCTTTTTCCTTT
TTGCACCGGTACGCTACGTTC-3′
and the second set being
5′-GCCGCAAAAAGTACAAAAAGGCTCGACGCCGCAGCTCGTCCTAGCTA
GCGTCGTAGCGTCTTAAG-3′
and
5′-CGACTTAAGACGCTACGACGCTAGCTAGGACGAGCTGCGGCGTCGAG
CCTTTTTGTACTTTTTGC-3′

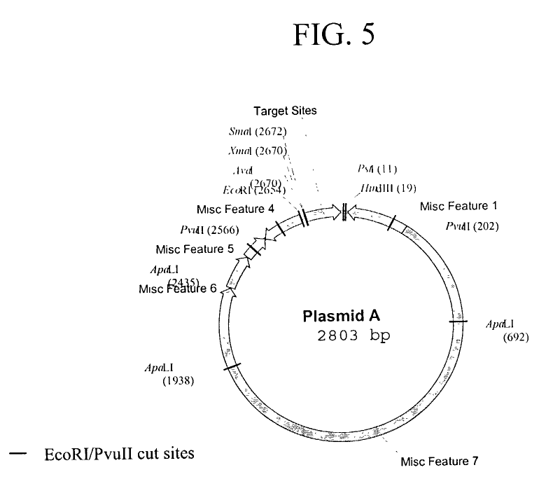

and ligating the resulting duplexes to the large pUC19 AvaI/SalI restriction fragment. A map of Plasmid A is shown in FIG. 5.

Plasmid B was prepared by hybridizing two sets of 5′-phosphorylated complementary oligoniucleotides, the first set being

the first set being
5′-CTAGATGCCGCTAAGTACTATGCCGCTAACTACTATGCCGCTAATTA
CTATGCCGC-3′
and
5′-CATAGTAATTAGCGGCATAGTAGTTAGCGGCATAGTACTTAGCGGC
AT-
and the second set being
5′-TAAATACTATGCCGCTAACTAGTATGCCGCTATGCA-3′
and
5′-TAGCGGCATACTAGTTAGCGGCATAGTATTTAGCGG-3′

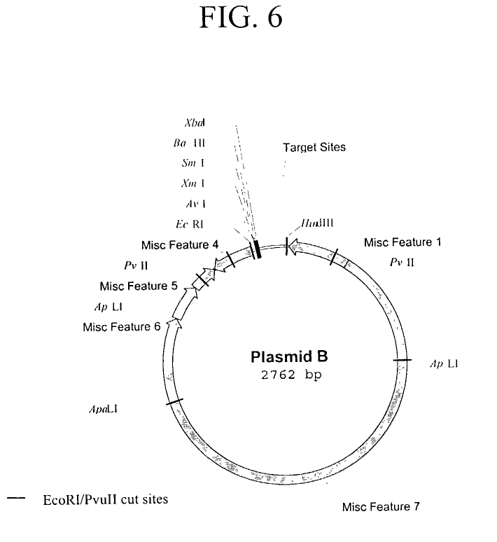

and ligating the resulting duplexes to the large pUC19 XbaI/PstI restriction fragment. A map of Plasmid B is shown in FIG. 6.

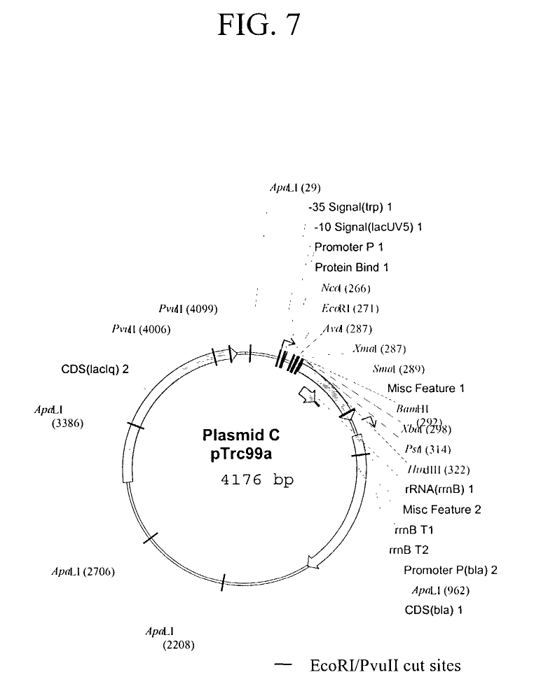

Plasmid C was the plasmid pTrc99a, obtained from Amersham Pharmacia Biotech, Inc. A map of Plasmid C is shown in FIG. 7.

The 3′-P32 end-labeled EcoRI/PvuII fragments from each plasmid were prepared by digesting the plasmids with EcoRI and PvuII with simultaneous fill-in using Sequenase v. 2.0, [alpha-P32]-deoxyadenosine-5′-triphosphate, and [alpha-P32]-thymidine-5′-triphosphate, and isolating the cloned fragments by nondenaturing gel electrophoresis. A and G sequencing reactions were carried out as described (See Maxam and Gilbert, Methods Enzymol., 1980, 65, 499-560; Iverson and Dervan, Methods Enzymol., 1987, 15, 7823-7830; Sambrook et al., 1989, Molecular Cloning, 2^{nd} ed., Cold Spring Harbor Laboratory Press: Cold Spring Harbor, N.Y.) Standard methods were used for all DNA manipulations (Sambrook et al., ibid.) The cut sites for EcoRI and PvuII are indicated by bars in the respective maps of Plasmids A, B, and C in FIGS. 5, 6, and 7.

The 351 base pair dsDNA restriction fragment (SEQ ID NO. IV) of Plasmid A contained the target sequences AAAAAGCAAAAA, AAAAAGACAAAAA, and AAAAAGTACAAAAA. The 310 base pair dsDNA restriction fragment (SEQ ID NO. V) of Plasmid B contained the target sequences AGTACT, AATACT, and ATTACT. The 352 base pair dsDNA restriction fragment (SEQ ID NO. VI) of Plasmid C contained the target sequences TGACAATTAAT, GACAATTAATCA, AATTAATCAT, ACAATTA, and ACAATTAAT. These fragments were used for quantitative DNase I footprinting experiments. Compounds used in the method of this invention bind to at least one of the target sites with a equal to or less than 100 nM, preferably equal to or less than 50 nM, and more preferably equal to or less than 20 nM. The target sequences were selected for the identity with, or similarity to, promoter sites for bacterial genes.

Quantitative DNase I footprint titration experiments were carried out as described previously (Dervan, WO 98/50582, 1998) with the following changes. All reactions were carried out in a total volume of 400 μL, with compound stock solution or water added to 15,000 cpm radiolabeled restriction fragment affording final solution conditions of 10 mM Tris HCl, 10 mM KCl, 10 mM MgCl₂, 5 mM CaCl₂, pH 7.0 and 0.01 nM, 0.1 nM, 1.0 nM, 10.0 nM compound or no compound for reference lanes. The compounds were allowed to equilbrate at 22° C. for 16 h. Footprinting reactions were initiated with addition of 10 μL of a DNase I stock solution (at the appropriate concentration to give ˜50% intact DNA) containing 1 mM DTT and allowed to proceed for 7 min at 22° C. The reactions were stopped, ethanol precipitated, resuspended in loading buffer, heat denatured, and placed on ice as described previously (Dervan WO 98/50582, 1998). The reaction products were separated on a precast 8% polyacrylamide denaturing sequencing Castaway gel with 32 preformed wells from Stratagene in 1×TBE at 2000 V. Gels were dried according to the manufacturer and exposed to a storage phosphor screen (Molecular Dynamics). Quantitation and data analysis were carried out as described in Dervan, WO 98/50582, 1998.

dsDNA binding results are provided in Table V:

TABLE V
dsDNA Binding

| | | | |
|---|---|---|---|
| | | | Target |
| | | Dissociation | Location |
| | Target | Constant | (Fragment- |
| Compound | Sequence | K_{d} (nM) | Plasmid). |

| | | | |
|---|---|---|---|
| 1 | AAAAAGCAAAAA | 0.01 | 351 bpA |
| 1 | AAAAAGACAAAAA | 0.01 | 351 bpA |
| 1 | AAAAAGTACAAAAA | 0.01 | 351 bpA |
| 1 | TGACAATTAAT | 2 | 352 bpC |
| 2 | AAAAAGTACAAAAA | 0.2 | 351 bpA |
| 2 | TGACAATTAAT | 10 | 352 bpC |
| 3 | AAAAAGTACAAAAA | 0.01 | 351 bpA |
| 3 | TGACAATTAAT | 2 | 352 bpC |
| 4 | AAAAAGTACAAAAA | 0.01 | 351 bpA |
| 4 | TGACAATTAAT | 10 | 352 bpC |
| 5 | AAAAAGTACAAAAA | 0.01 | 351 bpA |
| 5 | GACAATTAATCA | 2 | 352 bpC |
| 6 | AATTAATCAT | 20 | 352 bpC |
| 7 | ACAATTA | 2 | 352 bpC |
| 8 | AATTAATCAT | 0.2 | 352 bpC |
| 9 | GACAATTAATCA | 0.1 | 352 bpC |
| 10 | AATACT | 5 | 310 bpB |
| 10 | AATTAATCAT | 1 | 352 bpC |
| 11 | GACAATTAATCA | ≦1 | 352 bpC |
| 12 | GACAATTAATCA | ≦0.1 | 352 bpC |
| 13 | ATTACT | 5 | 310 bpB |
| 13 | AATTAATCAT | 10 | 352 bpC |
| 14 | AGTACT | 50 | 310 bpB |
| 14 | ACAATTAAT | 5 | 352 bpC |
| 15 | AGTACT | 50 | 310 bpB |
| 15 | AATTAATCAT | 2 | 352 bpC |
| 16 | ATTACT | 50 | 310 bpB |
| 16 | AATTAATCAT | 10 | 352 bpC |
| 17 | AGTACT | <100 | 310 bpB |
| 17 | AATTAATCAT | 5 | 352 bpC |
| 18 | ATTACT | 50 | 310 bpB |
| 18 | AATTAATCAT | 5 | 352 bpB |
| 19 | AGTACT | 50 | 310 bpB |
| 19 | ACAATTAAT | 1 | 352 bpC |
| 20 | AGTACT | 10 | 310 bpB |
| 20 | AATTAATCAT | 5 | 352 bpC |

What is claimed is:

1. A method for treating an infection by Gram-positive bacteria in a mammal, said method comprising administering to said mammal an effective amount of a compound that binds noncovalently in the minor groove of duplex DNA, which compound:
i) binds with a dissociation constant of equal to or less than 100 nM to at least one of:
(a) a target sequence AAAAAGCAAAAA in the 351 base pair EcoRI/PvuII restriction fragment of a polynucleotide of SEQ ID NO. I;
(b) a target sequence AAAAAGACAAAAA in the 351 base pair EcoRI/PvuII restriction fragment of a polynucleotide of SEQ ID NO. I;
(c) a target sequence AAAAAGTACAAAAA in the 351 base pair EcoRI/PvuII restriction fragment of a polynucleotide of SEQ ID NO. I;
(d) a target sequence AGTACT in the 310 base pair EcoRI/PvuII restriction fragment of a polynucleotide of SEQ ID NO. II;
(e) a target sequence AATACT in the 310 base pair EcoRI/PvuII restriction fragment of a polynucleotide of SEQ ID NO. II;
(l) a target sequence ATTACT in the 310 base pair EcoRI/PvuII restriction fragment of a polynucleotide of SEQ ID NO. II;
(g) a target sequence TGACAATTAAT in the 352 base pair EcoRI/PvuII restriction fragment of a polynucleotide of SEQ ID NO. III;
(h) a target sequence GACAATTAATCA in the 352 base pair EcoRI/PvuII restriction fragment of a polynucleotide of SEQ ID NO. III;
(i) a target sequence AATTAATCAT in the 352 base pair EcoRI/PvuII restriction fragment of a polynucleotide of SEQ ID NO. III;
(j) a target sequence ACAATTA in the 352 base pair EcoRI/PvuII restriction fragment of a polynucleotide of SEQ ID NO. III; and
(k) a target sequence ACAATTAAT in the 352 base pair EcoRI/PvuII restriction fragment of a polynucleotide of SEQ ID NO. III;
ii) exhibits a MIC of less than or equal to 2 μg/mL against at least one of Enterococcus faecium ATCC 51559, Staphylococcus aureus ATCC 27660, Staphylococcus aureus ATCC 33591, Staphylococcus aureus ATCC 43300, and Streptococcus pneumoniae ATCC 51422;
iii) exhibits a MIC of greater than or equal to 32 μg/mL against Candida albicans ATCC 38247; and
iv) has a molecular weight of from 100 to about 1100.

2. A method according to claim 1, wherein the dissociation constant is equal to or less than 50 nM.

3. A method in accordance with claim 1, wherein the dissociation constant is equal to or less than 20 nM.

4. A method in accordance with claim 1, wherein the compound has a MIC of equal to or less than 2 lug/mL against each of Enterococcus faecium ATCC 51559, Staphylococcus aureus ATCC 27660, Staphylococcus aureus ATCC 33591, Staphylococcus aureus ATCC 43300, and Streptococcus pneumoniae ATCC 51422.

5. A method in accordance with claim 1, wherein the compound has a MIC of equal to or less than 1 μg/mL against each of Enterococcus faecium ATCC 51559, Staphylococcus aureus ATCC 27660, Staphylococcus aureus ATCC ATCC 33591, Staphylococcus aureus ATCC 43300, and Streptococcus pneumoniae ATCC 51422.

6. A method in accordance with claim 1, wherein the compound has a molecular weight of from about 400 to about 800.

7. A method for treating an infection by Gram-positive bacteria in a mammal, said method comprising administering to said mammal an effective amount of a compound that binds noncovalently to duplex DNA, which compound
i) binds with a dissociation constant of equal to or less than 100 nM to at least one of:
(a) a target sequence AAAAAGCAAAAA in the 351 base pair EcoRI/PvuII restriction fragment of a polynucleotide of SEQ ID NO. I;
(b) a target sequence AAAAAGACAAAAA in the 351 base pair EcoRI/PvuII restriction fragment of a polynucleotide of SEQ ID NO. I;
(c) a target sequence AAAAAGTACAAAAA in the 351 base pair EcoRI/PvuII restriction fragment of a polynucleotide of SEQ ID NO. I;
(d) a target sequence AGTACT in the 310 base pair EcoRI/PvuII restriction fragment of a polynucleotide of SEQ ID NO. II;
(e) a target sequence AATACT in the 310 base pair EcoRI/PvuII restriction fragment of a polynucleotide of SEQ ID NO. II;
(f) a target sequence ATTACT in the 310 base pair EcoRI/PvuII restriction fragment of a polynucleotide of SEQ ID NO. II;
(g) a target sequence TGACAATTAAT in the 352 base pair EcoRI/PvuII restriction fragment of a polynucleotide of SEQ ID NO. III;
(h) a target sequence GACAATTAATCA in the 352 base pair EcoRI/PvuII restriction fragment of a polynucleotide of SEQ ID NO. III;
(i) a target sequence AATTAATCAT in the 352 base pair EcoRI/PvuII restriction fragment of a polynucleotide of SEQ ID NO. III;
(j) a target sequence ACAATTA in the 352 base pair EcoRI/PvuII restriction fragment of a polynucleotide of SEQ ID NO. III; and
(k) a target sequence ACAATTAAT in the 352 base pair EcoRI/PvuII restriction fragment of a polynucleotide of SEQ ID NO. III;
ii) has activity ratio X/Y equal to or greater than 16, wherein X is the MIC of the compound against Candida albicans ATCC 38247 and Y is the lowest MIC of the compound from among the MIC's for Enterococcus faecium ATCC 51559, Staphylococcus aureus ATCC 27660, Staphylococcus aureus 33591, Staphylococcus aureus ATCC 43300, and Streptococcus pneumoniae ATCC 51422; and
iii) has a molecular weight of from 100 to about 1100.

8. A method in accordance with claim 1, wherein X/Y is equal to or greater than 32.

9. A method in accordance with claim 7, wherein said compound binds with a dissociation constant of equal to or less than 100 nM to at least three of the target sequences in (a) through (k).

10. A method in accordance with claim 7, wherein said compound binds with a dissociation constant of equal to or less than 100 nM to at least five of the target sequences in (a) through (k).

11. A method in accordance with claim 7, wherein said compound binds with a dissociation constant of equal to or less than 100 nM to each of the target sequences in (a) through (k).

12. A method in accordance with claim 7, wherein said compound has a half-life of greater than four hours in plasma.

13. A method in accordance with claim 7, wherein said compound has the formula:
A-((L¹)ₚ-Ar¹)ₙ-L^{x}-B (I)
wherein
A is member selected from the group consisting of a substituted or unsubstituted aryl or heteroaryl group, a substituted or unsubstituted heterocyclic group, an amino group and a mono- or di-alkyl amino group;
the subscript n is an integer of from 2 to 7;
the subscript p in each instance is an integer of from 0 to 1, indicating the presence or absence of each linking group (L¹);
L¹ is a linking group in which the superscript i is an integer of from 1 to n, and each linking group can be the same or different from the other linking groups and is selected from the group consisting of-NH—, —NR—, —CONH—, —SO₂NH—, —CONR—, —SO₂NR—, (C₁-C₆)alkylene, (C₁-C₆)heteroalkylene, and combinations thereof in which each R is independently (C₁-C₆)alkyl;
Ar¹ is a substituted or unsubstituted aryl or heteroaryl group, in which the superscript i is an integer of from 1 to n and denotes the position away from A that is occupied by each aryl or heteroaryl group, and each Ar group can be the same or different from any other Ar group;
L^{x} is a linking group selected from —NH—, —NR—, —CONH—, —SO₂NH—, —CONR—, —SO₂NR—, (C₁-C₆)alkylene, (C₁-C₆)heteroalkylene, and combinations thereof in which each R is independently (C₁-C₆)alkyl; and
B is a member selected from the group consisting of a substituted or unsubstituted aryl or heteroaryl group, a substituted or unsubstituted heterocyclic group, an amino group and a mono- or di-alkyl amino group;
or a pharmaceutically acceptable salt, prodrug form or protected form thereof.

14. A method in accordance with claim 13, wherein said compound has a formula selected from the group consisting of:
```
A—L¹—Ar¹—L²—Ar²—L³—Ar³—L⁴—Ar⁴—L⁵—Ar⁵—L^{X}—B;
  A—L¹—Ar¹—L²—Ar²—Ar³13 L⁴—Ar⁴—L⁵—Ar⁵—L^{X}—B;
    A—L¹—Ar¹—L²—Ar²—L³—Ar³—L⁴—Ar⁴—L^{X}—B;
      A—L¹—Ar¹—L²—Ar²—L³—Ar³—L^{X}—B;
        A—L¹—Ar¹—L²—Ar²—L^{X}—B; and
          A—L¹—Ar¹—Ar²—L^{X}—B.
```

15. A method in accordance with claim 13, wherein said compound has the formula A-L¹-Ar¹-L²-Ar²L³-Ar³-L⁴-Ar⁴L^{x}-B.

16. A method in accordance with claim 13, wherein said compound has the formula A-L¹-Ar¹-L²-Ar-L³-Ar³-L^{x}-B.

17. A method in accordance with claim 13, wherein said compound binds in the minor groove of a DNA duplex comprising an AT-rich sequence selected from the group consisting of the target sequences (a) through (t).

18. A method in accordance with claim 13, wherein said compound binds in the minor groove of a DNA duplex comprising an AT-rich sequence selected from the group consisting of the target sequences (g) through (k).

19. A method in accordance with claim 13, wherein said compound is soluble in water at pH 7.5 in an amount greater than or equal to 0.1 mg/mL.

20. A method in accordance with claim 1, wherein said compound exhibits less than or equal to 90% protein binding in an in vitro protein binding assay.

21. A compound useful for the treatment of an infection by Gram-positive bacteria, said compound having the formula:
A-((L¹)ₚ-Ar¹)ₙ-L¹-B (I)
wherein
A is member selected from the group consisting of a substituted or unsubstituted aryl or heteroaryl group, a substituted or unsubstituted heterocyclic group, an amino group and a mono- or di-alkyl amino group;
the subscript n is an integer of from 2 to 7;
the subscript p in each instance is an integer of from 0 to 1, indicating the presence or absence of each linking group (L¹);
L¹ is a linking group in which the superscript i is an integer of from 1 to n, and each linking group can be the same or different from the other linking groups and is selected from the group consisting of —NH—, —CONH—, —SO₂NH—, —CONR—, —SO₂NR—, (C₁-C₆)alkylene, (C₁-C₆)heteroalkylene, and combinations thereof;
Arⁱ is a substituted or unsubstituted aryl or heteroaryl group, in which the superscript i is an integer of from 1 to n and denotes the position away from A that is occupied by each aryl or heteroaryl group, and each Ar group can be the same or different from any other Ar group;
L^{x} is a linking group selected from —CONH—, —SO₂NH—, —CONR—, —SO₂NR—, (C₁-C₆)alkylene, (C₁-C₆)heteroalkylene, and combinations thereof; and
B is a member selected from the group consisting of a substituted or unsubstituted aryl or heteroaryl group, a substituted or unsubstituted heterocyclic group, an amino group and a mono- or di-alkyl amino group;
or a pharmaceutically acceptable salt, prodrug form or protected form thereof; wherein said compound
i) binds with a dissociation constant of equal to or less than 100 nM to at least one of:
(a) a target sequence AAAAAGCAAAAA in the 351 base pair EcoRI/PvuII restriction fragment of a polynucleotide of SEQ ID NO. I;
(b) a target sequence AAAAAGACAAAAA in the 351 base pair EcoRI/PvuII restriction fragment of a polynucleotide of SEQ ID NO. I;
(c) a target sequence AAAAAGTACAAAAA in the 351 base pair EcoRI/PvuII restriction fragment of a polynucleotide of SEQ ID NO. I;
(d) a target sequence AGTACT in the 310 base pair EcoRI/PvuII restriction fragment of a polynucleotide of SEQ ID NO. II;
(e) a target sequence AATACT in the 310 base pair EcoRI/PvuII restriction fragment of a polynucleotide of SEQ ID NO. II;
(f) a target sequence ATTACT in the 310 base pair EcoRI/PvuII restriction fragment of a polynucleotide of SEQ ID NO. II;
(g) a target sequence TGACAATTAAT in the 352 base pair EcoRI/PvuII restriction fragment of a polynucleotide of SEQ ID NO. III;
(h) a target sequence GACAATTAATCA in the 352 base pair EcoRI/PvuII restriction fragment of a polynucleotide of SEQ ID NO. III;
(i) a target sequence AATTAATCAT in the 352 base pair EcoRI/PvuII restriction fragment of a polynucleotide of SEQ ID NO. III;
(j) a target sequence ACAATTA in the 352 base pair EcoRI/PvuII restriction fragment of a polynucleotide of SEQ ID NO. III; and
(k) a target sequence ACAATTAAT in the 352 base pair EcoRI/PvuII restriction fragment of a polynucleotide of SEQ ID NO. III;
ii) has activity ratio X/Y equal to or greater than 16, wherein X is the MIC of the compound against Candida albicans ATCC 38247 and Y is the lowest MIC of the compound from among the MIC's for Enterococcus faecium ATCC 51559, Staphylococcus aureus ATCC 27660, Staphylococcus aureus 33591, Staphylococcus aureus ATCC 43300, and Streptococcus pneumoniae ATCC 51422; and
iii) has a molecular weight of from 100 to about 1100.

22. A compound in accordance with claim 21, having a formula selected from the group consisting of:
```
A—L¹—Ar¹—L²—Ar²—L³—Ar³—L⁴—Ar⁴—L⁵—Ar⁵—L^{X}—B;
  A—L¹—Ar¹—L²—Ar²—Ar³—L⁴—Ar⁴—L⁵—Ar⁵—L^{X}—B;
    A—L¹—Ar¹—L²—Ar²—L³—Ar³—L⁴—Ar⁴—L^{X}—B;
      A—L¹—Ar¹—L²—Ar²—L³—Ar³—L^{X}—B;
        A—L¹—Ar¹—L²—Ar²—L^{X}—B; and
          A—L¹—Ar¹—Ar²—L^{X}—B.
```

23. A compound in accordance with claim 21, wherein each Ar is independently selected from the group consisting of substituted and unsubstituted thienyl, substituted and unsubstituted thiazolyl, substituted and unsubstituted isothiazolyl, substituted and unsubstituted imidazolyl, substituted and unsubstituted pyrrolyl, substituted and unsubstituted oxazolyl, substituted and unsubstituted triazolyl, substituted and unsubstituted isoquinolyl, substituted and unsubstituted pyrazolyl, substituted and unsubstituted benzothienyl, substituted and unsubstituted pyrazinyl, substituted and unsubstituted pyradinyl and substituted and unsubstituted phenyl.

24. A compound in accordance with claim 21, wherein each Ar is independently selected from the group consisting of substituted and unsubstituted thienyl, substituted and unsubstituted thiazolyl, substituted and unsubstituted isothiazolyl, substituted and unsubstituted imidazolyl, substituted and unsubstituted isoquinolyl, substituted and unsubstituted pyrazolyl, substituted and unsubstituted benzothienyl, substituted and unsubstituted pyrazinyl, substituted and unsubstituted pyradinyl and substituted and unsubstituted pyrrolyl; and each Y is independently selected from the group consisting of —C(O)—, —NEC(O)— and —C(O)NH—.

25. A compound in accordance with claim 21, wherein said compound has a molecular weight of from 100 to 750.

26. The use of a compound of claim 21 for the preparation of a medicament useful for the treatment of an infection by Gram-positive bacteria in a mammal.
